(19) United States
(12) Patent Application Publication
(10) Pub. No.: US 2012/0197356 A1
(43) Pub. Date: Aug. 2, 2012

(54) Waveforms for Remote Electrical Stimulation Therapy

(75) Inventors: Xuan Wei, Plymouth, MN (US); Duane L. Bourget, Albertville, MN (US)

(73) Assignee: Medtronic, Inc., Minneapolis, MN (US)

(21) Appl. No.: 13/355,122

(22) Filed: Jan. 20, 2012

Related U.S. Application Data
(60) Provisional application No. 61/436,910, filed on Jan. 27, 2011.

(51) Int. Cl. A61N 1/36

(52) U.S. Cl. 607/74

(57) ABSTRACT

Electrical stimulation systems and methods are configured to deliver remote electrical stimulation to a patient. Stimulation waveforms are employed that are designed to penetrate tissue within a patient to transmit the electrical stimulation from an origination site to a remote delivery site. The waveforms are defined by a series of pulses, which are characterized by a number of parameters, including pulse width, pulse frequency, constant voltage or constant current amplitude, and electrode polarity (anode or cathode). The waveforms include an envelope electrical stimulation pulse train including charge balanced pulses modulated by a high frequency carrier signal configured to deeply penetrate patient tissue to carry the electrical pulse train from an origination site to a remote delivery site.

This application claims the benefit of U.S. Provisional Patent Application No. 61/436,910, filed Jan. 27, 2011, the entire content of which is incorporated herein by this reference.

### BACKGROUND

A variety of medical devices are used for chronic, e.g., long-term, delivery of therapy to patients suffering from conditions that range from chronic pain, tremor, Parkinson's disease, and epilepsy, to urinary or fecal incontinence, sexual dysfunction, obesity, spasticity, and gastroparesis. As an example, electrical stimulation generators are used for chronic delivery of electrical stimulation therapies such as neurostimulation, muscle stimulation, target organ stimulation, or the like. Electrical stimulation may be delivered in the form of series of electrical pulses that form a stimulation waveform that may be characterized by a number of the different shapes and forms. Typically, such devices provide therapy continuously or periodically according to parameters contained within a program. A program may comprise respective values for each parameter in a set of therapeutic parameters specified by a clinician. For example, a program may define characteristics of the electrical pulses defining the stimulation waveform, including pulse width, pulse frequency, constant voltage or constant current amplitude, and electrode polarity (anode or cathode).

### SUMMARY

This disclosure is directed to medical devices, systems, and techniques for delivering electrical stimulation therapy to treat one or more patient conditions. A medical device may deliver electrical stimulation therapy, which includes a series of electrical stimulation pulses, via one or more electrodes to one or more tissue sites of a patient. The series of electrical stimulation pulses may be delivered to the one or more tissues sites in a manner that effectively treats the patient condition. In some examples, the medical device may be configured to generate and deliver electrical stimulation therapy, e.g., pelvic floor stimulation, to a patient using one or more example waveforms described in this disclosure.

In one example, a method includes generating a series of electrical pulses including a first frequency and alternating pulse polarities, modulating the series of electrical pulses with a carrier signal including a second frequency such that each pulse in the series of electrical pulses includes a series of carrier pulses comprising a single polarity and the second frequency, and delivering the series of electrical pulses modulated by the carrier signal from an origination site to a remote delivery site within a patient. The first frequency is configured to produce a therapeutic effect and the second frequency is configured to enable the series of electrical pulses to penetrate tissue between the origination site and the remote delivery site. The series of electrical pulses is substantially charge balanced.

In another example, a system includes a stimulation generator and a processor. The stimulation generator is configured to generate and deliver electrical pulses from an origination site to a remote delivery site within a patient. The processor is configured to control the stimulation generator to generate and deliver a series of electrical pulses including a first frequency and alternating pulse polarities and modulate the series of electrical pulses with a carrier signal including a second frequency such that each pulse in the series of electrical pulses includes a series of carrier pulses comprising a single polarity and the second frequency. The first frequency is configured to produce a therapeutic effect and the second frequency is configured to enable the series of electrical pulses to penetrate tissue between the origination site and the remote delivery site. The series of electrical pulses is substantially charged balanced.

In another example, a non-transitory computer-readable storage medium comprising instructions to cause a programmable processor to control a stimulation generator to generate a series of electrical pulses comprising a first frequency and alternating pulse polarities, modulate the series of electrical pulses with a carrier signal comprising a second frequency such that each pulse in the series of electrical pulses comprises a series of carrier pulses comprising a single polarity and the second frequency, and deliver the series of electrical pulses modulated by the carrier signal from an origination site to a remote delivery site within a patient. The first frequency is configured to produce a therapeutic effect and the second frequency is configured to enable the series of electrical pulses to penetrate tissue between the origination site and the remote delivery site. The series of electrical pulses is substantially charged balanced.

In another example, a medical system includes means for generating a series of electrical pulses comprising a first frequency and alternating pulse polarities, means for modulating the series of electrical pulses with a carrier signal comprising a second frequency such that each pulse in the series of electrical pulses comprises a series of carrier pulses comprising a single polarity and the second frequency, and means for delivering the series of electrical pulses modulated by the carrier signal from an origination site to a remote delivery site within a patient. The first frequency is configured to produce a therapeutic effect and the second frequency is configured to enable the series of electrical pulses to penetrate tissue between the origination site and the remote delivery site. The series of electrical pulses is substantially charge balanced.

The details of one or more examples are set forth in the accompanying drawings and the description below. Other features, objects, and advantages of examples according to this disclosure will be apparent from the description and drawings, and from the claims.

### BRIEF DESCRIPTION OF DRAWINGS

FIG. 1 is a schematic diagram illustrating an example implantable electrical stimulation system.

FIG. 2 is a schematic diagram illustrating another example implantable electrical stimulation system.

FIG. 3 is a block diagram illustrating example components of an implantable electrical stimulator that delivers electrical stimulation therapy to a patient.

FIG. 4 is a block diagram illustrating example components of an external programmer that receives user input and communicates with an electrical stimulator.

FIGS. 5A-5C illustrate a series of charge balanced electrical pulses modulated by a high frequency carrier signal.

FIG. 6 is a plot illustrating an example waveform by which electrical stimulation may be delivery to a patient.

FIG. 7 is a plot illustrating an example waveform representing an example series of electrical stimulation pulses for delivery to a patient.

FIGS. 8A and 8B are plots illustrating two other example waveforms representing example series of electrical stimulation pulses for delivery to a patient.

FIG. 9 is a plot illustrating another example waveform representing an example series of electrical stimulation pulses for delivery to a patient.

FIG. 10 is a plot illustrating another example waveform representing an example series of electrical stimulation pulses for delivery to a patient.

FIG. 11 is a block diagram illustrating an example stimulation generator for delivery of electrical stimulation therapy.

FIG. 12 is a flow chart illustrating an example method of generating and delivering a series of charge balanced electrical pulses modulated by a high frequency carrier signal to a patient.

### DETAILED DESCRIPTION

This disclosure is directed to medical devices, systems, and techniques for delivery of electrical stimulation therapy to treat one or more patient conditions. A medical device may deliver electrical stimulation therapy including a series of electrical stimulation pulses via one or more electrodes to one or more tissue sites of patient. The series of electrical stimulation pulses may be delivered to the one or more tissue sites in a manner that effectively treats the patient condition. Example waveforms are described which represent example series of electrical stimulation pulses that may be delivered to a patient for electrical stimulation therapy.

In some examples, electrical stimulation systems may be used to deliver electrical stimulation therapy to patients to treat a variety of patient symptoms or conditions, such as pelvic floor disorders of patients. Pelvic floor disorders may include urinary incontinence (e.g., stress incontinence or urge incontinence) or other bladder dysfunction, fecal incontinence, pelvic pain, bowel dysfunction, and sexual dysfunction. Although the following examples are described in the context of pelvic floor stimulation, examples according to this disclosure may be applied to a variety of locations in the body of a patient to treat a variety of conditions, including, e.g. gastric stimulation, spinal cord stimulation (SCS), peripheral nerve stimulation (PNS), and/or peripheral nerve field stimulation (PNFS).

The effectiveness of the electrical stimulation therapy in treating a patient disease or disorder can depend on one or more properties of the electrical stimulation energy generated and delivered from a stimulator to the patient. For example, values or pulse width, pulse frequency, constant voltage or constant current amplitude, and electrode polarity (anode or cathode) may be defined for a series of electrical stimulation pulses delivered to a patient to treat a disorder or disease, in addition to microduty and/or macroduty cycles for the stimulation therapy. The relationship between each of these parameter values may be expressed as a waveform, e.g., the waveform defined by the series of electrical stimulation pulses plotted in terms of amplitude (controlled current or controlled voltage) versus time. In some examples, the delivery of electrical stimulation therapy in a manner consistent with one or more particular electrical stimulation waveforms may be utilized to effectively treat one or more patient diseases or disorders using electrical stimulation therapy.

In many applications of electrical stimulation therapy, the location from which the electrical pulses defining the stimulation originate and the target delivery site at which the stimulation is directed at some structure within the body of a patient, e.g. a particular nerve, such as a sacral nerve are coincident or very close to one another. For example, stimulation may be delivered by one or more electrodes connected to a medical lead. In such cases, the electrodes are commonly arranged at or very close to the target delivery site within the patient. In the following examples, however, techniques are described for delivering remote electrical stimulation, in which the electrical pulses defining the stimulation are delivered to a delivery site that is remote from site at which the pulses originate. As used in this disclosure, remote delivery site may refer to sites that are more than 1 centimeter (cm) from the electrical stimulation origination site. The origination site may refer to the location or region from which the remote electrical stimulation, e.g. pulse train of electrical pulses originates. For example, the origination site may be the location or region where one or more electrodes delivering stimulation to a patient are arranged.

The following examples disclose various mechanisms by which remote electrical stimulation may be accomplished. In general, however, examples according to this disclosure employ stimulation waveforms that are specifically designed to penetrate tissue within a patient to transmit the electrical stimulation from the origination site to a remote delivery site. The disclosed waveforms are defined by a series of electrical stimulation pulses, which are characterized by a number of parameters, including pulse width, pulse frequency constant voltage or constant current amplitude, and electrode polarity (anode or cathode). The series of pulses employed in examples according to this disclosure include an envelope stimulation pulse train modulated by a high frequency carrier signal configured to deeply penetrate patient tissue to carry the stimulation pulse train from an origination site to a remote delivery site.

In addition to delivering stimulation from an origination site to a remote delivery site, examples according to this disclosure deliver electrical stimulation therapy to a patient such that the electrical stimulation energy is charge balanced. As used herein, charge balanced may generally refer to the property of the net charge of one or more stimulation pulses being approximately equal to zero. For example, when a pair of single phase pulses having opposite polarity or a coupled pulse pair in a biphasic electrical pulse are substantially charge balanced, the charge of the first pulse substantially offsets the charge of the second pulse such that the net charge of the pulses is substantially zero. Graphically, in terms of two pulses having opposite polarity, charge balance implies that the area between the amplitude curve and the zero amplitude line for a first pulse having a first polarity is equal to the area between the amplitude curve and the zero amplitude line for the second pulse having the opposite polarity. In general, charge balance may be desirable for limiting electrochemical reactions on the surface of stimulation electrodes that can cause corrosion of the electrodes, formation of noxious compounds at the stimulation site, and transfer of electrode material into the surrounding tissue. Although remote stimulation is directed at a target delivery site that is remote from the origination site, tissue damage is still possible in the proximity of the electrodes, i.e. at the origination site and thus charge balancing is beneficial in remote stimulation applications.

Combining high frequency carrier signal modulation and charge balancing, examples according to this disclosure deliver electrical stimulation defined by a waveform that includes successive electrical pulses of alternating polarity, each of which is modulated by a high frequency carrier signal and substantially charge balanced with the other pulse. In one example, a method includes delivering a series of electrical pulses including a first frequency and alternating pulse polarities from an origination site to a remote delivery site within a patient. The series of electrical pulses is modulated with a carrier signal including a second frequency such that each pulse in the series of electrical pulses includes a series of carrier pulses including a single polarity and the second frequency. The first frequency is configured to produce a therapeutic effect and the second frequency is configured to enable the series of electrical pulses to penetrate tissue between the origination site and the remote delivery site. Each pair of successive pulses in the series of electrical pulses is substantially charge balance.

The various techniques and features described in this disclosure may be implemented within an external programmer, an external or implantable electrical stimulator, or a combination of both. The external programmer may be a external programmer that accompanies a patient through a daily routine. Various examples of programmers, stimulators and associated functionality are provided for illustration, but without limitation of the various aspects of the disclosure as broadly embodied and described herein.

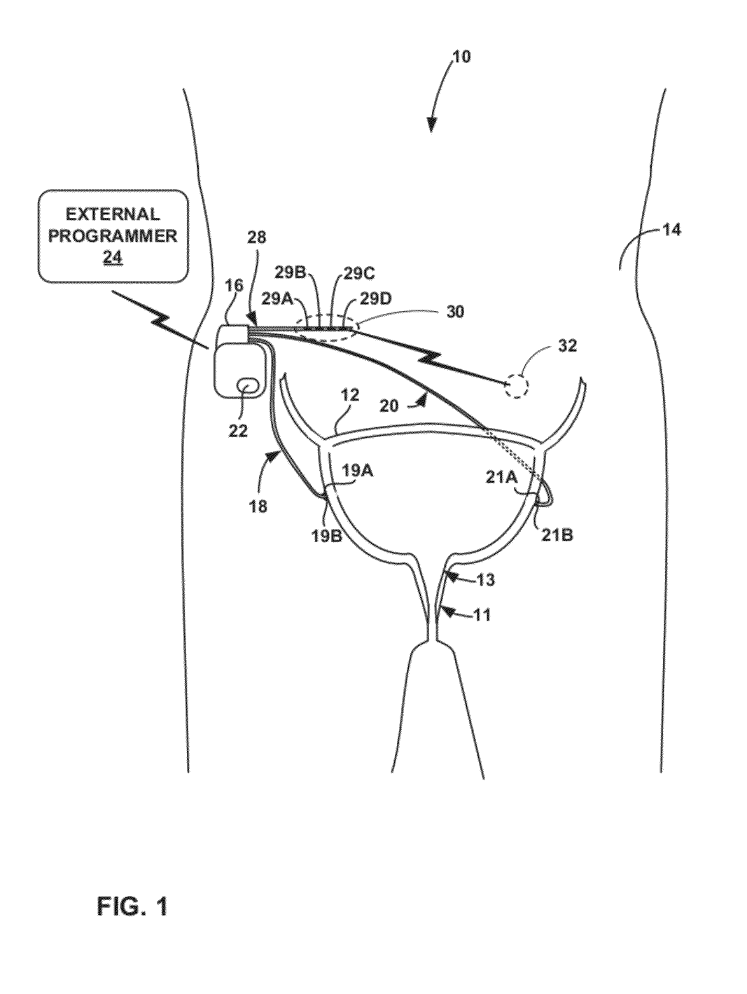

FIG. 1 is a conceptual diagram that illustrates an example of a therapy system 10 that delivers electrical stimulation therapy to modulate dysfunction of bladder 12 of patient 14, Patient 14 may be a human or non-human patient. Therapy system 10 includes an implantable medical device (MID) 16, which is coupled to leads 18, 20, and 28 and sensor 22. System 10 also includes an external programmer 24, which communicates with BID 16 via a wireless communication protocol. IMD 16 generally operates as a therapy device that delivers electrical stimulation to, for example, a target tissue site proximate a spinal nerve, a sacral nerve, a pudendal nerve, dorsal genital nerve, a tibial nerve, an inferior rectal nerve, a perineal nerve, or branches of any of the aforementioned nerves. IMD 16 provides electrical stimulation therapy to patient 14 by generating and delivering a programmable electrical stimulation signal (e.g., in the form of electrical pulses or a series of electrical pulses in an electrical waveform) from origination site 30 near lead 28 and, more particularly, near electrodes 29A-29D (collectively referred to as “electrodes 29”) to remote delivery site 32 near a target nerve.

IMD 16 may be surgically implanted in patient 14 at any suitable location within patient 14, such as near the pelvis. In some examples, IMD 16 may be implanted in a subcutaneous location in the side of the lower abdomen or the side of the lower back or upper buttocks. IMD 16 has a biocompatible housing, which may be formed from titanium, stainless steel, a liquid crystal polymer, or the like. The proximal ends of leads 18, 20, and 28 are both electrically and mechanically coupled to IMD 16 either directly or indirectly, e.g., via respective lead extensions. Electrical conductors disposed within the lead bodies of leads 18, 20, and 28 electrically connect sense electrodes (e.g., electrodes 19A, 19B, 21A, and 21B) and stimulation electrodes, such as electrodes 29, to a sensing module and a therapy delivery module (e.g., a stimulation generator) within IMD 16. In the example of FIG. 1, leads 18 and 20 carry electrodes 19A, 199 (collective referred to as “electrodes 19”) and electrodes 21A, 21B (collectively referred to as “electrodes 21”), respectively. As described in further detail below, electrodes 19 and 21 may be positioned for sensing an impedance of bladder 12, which may increase as the volume of urine within bladder 12 increases.

In some examples, IMD 16 may be a leadless implantable device that includes electrodes on a housing of the device. For example, IMD 16 may include at least two individual electrodes to deliver the stimulation to remote delivery site 32 from the implantation location of the IMD. In some examples, the housing of IMD 16 may act as one electrode, where at least one non-housing electrode can be an electrically isolated electrode referenced to the housing of IMD 16 to deliver stimulation. IMD 16 may be secured within patient 14 using any suitable attachment technique, including screwing-in, hooking and clamping of IMD 16 to tissue of the patient. A leadless IMD 16 may deliver stimulation to patient 14 according to one or more example high frequency carrier signal modulated and charge balanced waveforms described in the disclosure.

One or more medical leads, e.g., leads 18, 20, and 28, may be connected to IMD 16 and surgically or percutaneously tunneled to place one or more electrodes carried by a distal end of the respective lead at a target site. For example, lead 28 may be positioned at origination site 30 such that electrodes 29 deliver stimulation therapy defined by high frequency carrier signal modulated and charge balanced waveforms to a remote delivery site, e.g. remote site 32, or, in other examples, to a spinal, sacral or pudendal nerve, e.g. to reduce a frequency of contractions of bladder 12. In other examples, lead 28 may also deliver stimulation therapy from origination site 30 to a hypogastric nerve, a pudendal nerve, a dorsal penile/clitoral nerve, the urinary sphincter, or any combination thereof to a promote closure of a urinary sphincter of patient 14. Electrodes 29 of the common lead 28 may deliver stimulation to the same or different nerves. In FIG. 1, leads 18 and 20 are placed proximate to an exterior surface of the wall of bladder 12 at first and second locations, respectively. In other examples of therapy system 10, IMD 16 may be coupled to more than one lead that includes electrodes for delivery of electrical stimulation to different stimulation sites within patient 14, e.g., to target different nerves.

In the example shown in FIG. 1, leads 18, 20, 28 are cylindrical. Electrodes 19, 20, 29 of leads 18, 20, 28, respectively, may be ring electrodes, segmented electrodes, partial ring electrodes or any suitable electrode configuration. Segmented and partial ring electrodes each extend along an arc less than 360 degrees (90-120 degrees) around the outer perimeter of the respective lead 18, 20, 28. In some examples, segmented electrodes 29 of lead 28 may be useful for targeting different fibers of the same or different nerves to generate different physiological effects for first and second stimulation therapies. In some examples, one or more of leads 18, 20, 28 may be, at least in part, paddle-shaped (e.g., a “paddle” lead), and may include an array of electrodes on a common surface, which may or may not be substantially flat. Additional electrode types may be employed for one or more of electrodes 19, 20, and 29, including, e.g. cuff electrodes.

The illustrated numbers and configurations of leads 18, 20, and 28 and electrodes carried by leads 18, 20, and 28 are merely exemplary. Other configurations, e.g., numbers and positions of leads and electrodes are also contemplated. For example, in other implementations, IMD 16 may be coupled to additional leads or lead segments having one or more electrodes positioned at the same or different origination sites remote from delivery sites at or proximate to, e.g. the spinal cord or in the pelvic region of patient 14. The additional leads may be used for delivering different stimulation therapies to respective stimulation sites within patient 14 or for monitoring at least one physiological parameter of patient 14.

In examples according to this disclosure, IMD 16 is configured to deliver stimulation defined by a waveform designed to penetrate tissue within patient 14 to transmit the electrical stimulation from origination site 30 to remote delivery site 32. In one example, IMD 16 delivers electrical stimulation to patient 14 via electrodes 29, which is defined by a waveform that includes successive electrical pulses of alternating polarity, each of which is modulated by a high frequency carrier signal and substantially charge balanced with the other pulse. For example, IMD 16 may be configured to deliver a series of electrical pulses including a first frequency and alternating pulse polarities from origination site 30 to remote delivery site 32 within patient 14. IMD 16 may modulate the series of electrical pulses with a carrier signal including a second frequency such that each pulse in the series of electrical pulses includes a series of carrier pulses. In one example, IMD 16 may be programmed to set the first frequency to produce one of a number of particular therapeutic effects, e.g. to modulate contraction of bladder 14. IMD 16 may also be programmed to set the second frequency to enable the series of electrical pulses to penetrate tissue of patient 14 between origination site 30 and remote delivery site 32. IMD 16 may deliver a series of electrical pulses to patient 14, in which each pair of successive pulses in the series of electrical pulses is substantially charge balance.

In some examples, IMD 16 may deliver stimulation therapy to patient 14 in an open loop manner according to one or more stimulation programs without input from a feedback loop, e.g., that varies as a function of the response of patient 14 to the therapy. In some examples, IMD 16 may deliver the stimulation therapy in a closed loop manner. For example, IMD 16 may sense activity of bladder 12, e.g., contractions during a time period prior to delivery of the stimulation therapy to establish a baseline contraction frequency of bladder 12, or the baseline contraction frequency may be stored in a memory of IMD 16 or another device (e.g., programmer 24). IMD 16 may sense contractions of bladder 12 via one or more means, such as, for example, electrodes 19 or 21, or sensor 22. IMD 16 may detect contractions of bladder 12 based on, for example, bladder impedance, bladder pressure, pudendal or sacral afferent nerve signals, a urinary sphincter EMG, or any combination thereof IMD 16 then may utilize the sensed contractions of bladder 12 to determine a baseline contraction frequency of bladder 12, e.g., as a number of contractions of bladder 12 per unit time. The baseline contraction frequency of bladder 12 may represent the patient state when no therapeutic effects from delivery of stimulation by IMD 16 are present. In some cases, however, patient 14 may also receive other types of therapy for managing bladder dysfunction, such as a pharmaceutical agent. The baseline contraction frequency of bladder 12 may represent the patient state when patient 14 is under the influence of the pharmaceutical agent but not electrical stimulation therapy.

In such an example, after determining a baseline contraction frequency, IMD 16 delivers stimulation therapy to patient 14 in an attempt to, e.g., relax the bladder by reducing the contraction frequency of the bladder, IMD 16 may sense, e.g., via electrodes 19 or 21 or sensor 22, a contraction frequency of bladder 12 while the IMD delivers stimulation to patient 14. In some examples, IMD 16 may sense a contraction frequency of bladder 12 periodically, e.g., once per minute. IMD 16 may compare the sensed contraction frequency of bladder 12 to the baseline contraction frequency or a threshold frequency that is based on the baseline contraction frequency. The threshold frequency may be less than the baseline contraction frequency. In some examples, when the sensed contraction frequency is within a certain value of the baseline contraction frequency or is above the threshold frequency, IMD 16 may continue delivering stimulation therapy to patient 14 in response to this feedback. In some examples, when the sensed contraction frequency is below a threshold contraction frequency, IMD 16 may cease stimulation therapy.

In some examples, IMD 16 may deliver stimulation therapy to patient 14 when, e.g., a function of bladder 12 exceeds a particular threshold. In one example, IMD 16 determines impedance through bladder 12, which varies as a function of the contraction of bladder 12, via electrodes 19 and 21 on leads 18 and 20, respectively. In the example of FIG. 1, IMD 16 also includes a sensor 22 for detecting changes in the contraction of bladder 12. Sensor 22 may include, fir example, a pressure sensor for detecting changes in bladder pressure, electrodes for sensing pudendal or sacral afferent nerve signals, electrodes for sensing urinary sphincter EMG signals (or anal sphincter EMG signals in examples in which therapy system 10 provides therapy to manage fecal urgency or fecal incontinence), or any combination thereof. In one example, sensor 22 may comprise a patient motion sensor that generates a signal indicative of patient activity level or posture state. In such an example, IMD 16 may control the delivery of stimulation therapy to patient 14 upon detecting a patient activity level exceeding a particular threshold based on the signal from the motion sensor. The patient activity level that is greater than or equal to a threshold (which may be stored in a memory of IMD 16) may indicate that there is an increase in the probability that an incontinence event will occur, and, therefore, the stimulation therapy may be desirable.

System 10 includes external programmer 24. In some examples, programmer 24 may be a wearable communication device, with activation of the stimulation therapy integrated into a key fob or a wrist watch, handheld computing device, computer workstation, or networked computing device. Programmer 24 may include a user interface that receives input from a user (e.g., patient 14, a patient caretaker or a clinician). In some examples, the user interface includes, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display. It should be noted that the user may also interact with programmer 24 and/or ICD 16 remotely via a networked computing device.

A clinician user, e.g. a physician, technician, surgeon, electrophysiologist, may also interact with programmer 24 or another separate programmer (not shown), such as a clinician programmer, to communicate with IMD 16. Such a user may interact with programmer 24 to retrieve physiological or diagnostic information from IMD 16, The user may also interact with programmer 24 to program IMD 16, e.g., select values for the stimulation parameter values with which IMD 16 generates and delivers stimulation and/or the other operational parameters of IMD 16. For example, the user may use programmer 24 to retrieve information from IMD 16 regarding the contraction of bladder 12 and/or voiding events. As another example, the user may use programmer 24 to retrieve information from IMD 16 regarding the performance or integrity of IMD 16 or other components of system 10, such as leads 18, 20, and 28, or a power source of IMD 16. In other examples, the user may employ programmer 24 to program IMD 16 to delivery stimulation therapy defined by high frequency carrier signal modulated and charge balanced waveforms, including programming values for pulse width, pulse frequency, constant voltage or constant current amplitude, and electrode polarity (anode or cathode).

IMD 16 and programmer 24 may communicate via various wireless communication techniques. Examples of communication techniques include low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

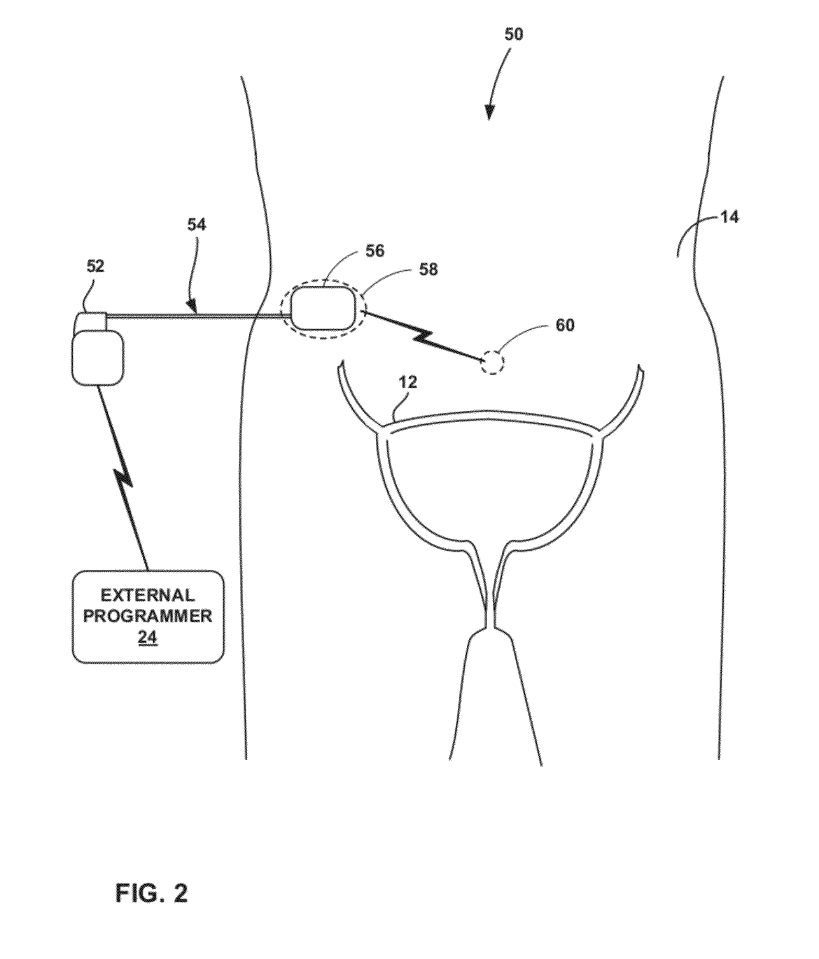

FIG. 2 is a conceptual diagram that illustrates another example of therapy system 50. Therapy system 50 may deliver electrical stimulation therapy defined by stimulation waveforms similar to those described above with reference to system 10 of FIG. 1, e.g. high frequency carrier signal modulated and charge balanced waveforms to modulate dysfunction of bladder 12 of patient 14. Therapy system 50, however, includes external stimulator 52, which is coupled to lead 54 and epidermal electrode pad 56. In the example of FIG. 2, epidermal electrode pad may include a number of electrodes, various combinations of which may be employed to deliver stimulation. In another example, however, system 50 may include multiple electrode pads, each of which may include one or more electrodes for delivering stimulation to patient 14. In the example of FIG. 2, external stimulator 52 is configured to deliver remote electrical stimulation to patient 14 via electrical stimulation pulses delivered by electrode pad 56 from origination site 58 outside of the body of the patient to remote delivery site 60 within the body, e.g. at or near one or more pelvic floor nerves.

In another example according to this disclosure, an external stimulator may be coupled to a percutaneous lead including electrodes arranged at the distal end of the lead within the body of a patient. In such an example, the stimulator may be configured to deliver remote electrical stimulation to the patient via electrical stimulation pulses delivered by the implanted electrodes at the distal end of the percutaneous lead from origination site within the body of the patient to a remote delivery site within the body, e.g. at or near one or more pelvic floor nerves.

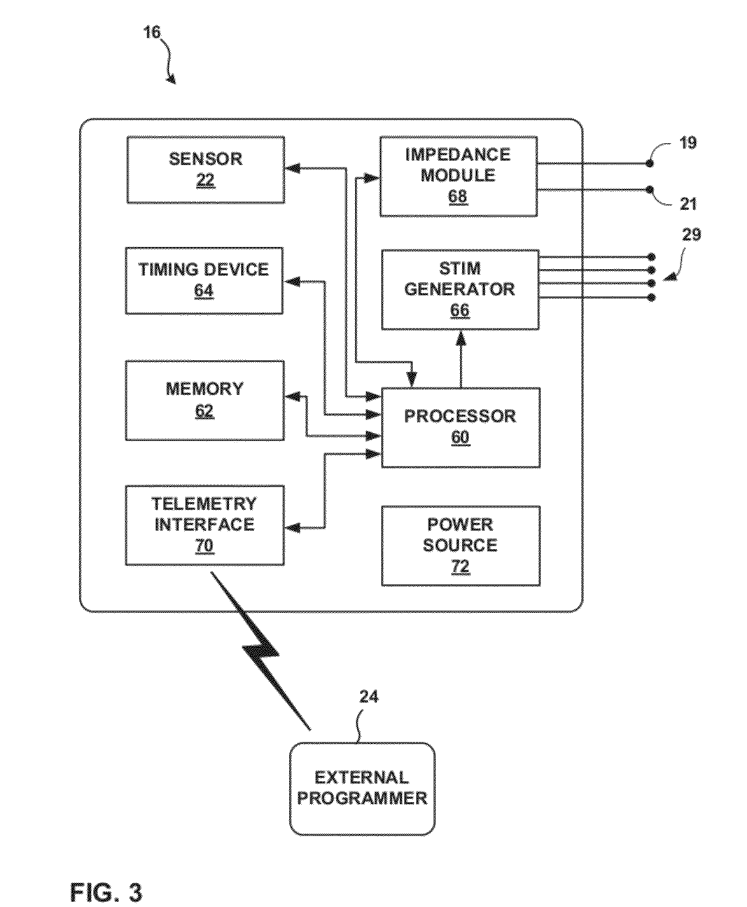

FIG. 3 is a block diagram illustrating example components of IMD 16 that delivers stimulation therapy to patient 14. In the example of FIG. 3, IMD 16 includes sensor 22, processor 60, memory 62, timing device 64, stimulation generator 66, impedance module 68, wireless telemetry interface 70 and power source 72. In some examples, IMD 16 may generally conform to the Medtronic Itrel 3 Neurostimulator, manufactured and marketed by Medtronic, Inc., of Minneapolis, Minn. However, the structure, design, and functionality of IMD 16 may be subject to wide variation without departing from the scope of the disclosure as broadly embodied and described in this disclosure.

IMD 16 may include any suitable arrangement of hardware, including, e.g., processor 60, alone or in combination with software and/or firmware, to perform the techniques attributed to IMD 16 and timing device 62, stimulation generator 66, impedance module 68, and telemetry interface 70 of IMD 16.

Processor 60 controls stimulation generator 66 by setting and adjusting stimulation parameters such as pulse amplitude, pulse rate, pulse width and duty cycle, in the case that stimulation generator 66 generates pulses. Alternative examples may direct stimulation generator 66 to generate continuous electrical signals, e.g., a sine wave. Processor 60 may be responsive to parameter adjustments or parameter sets received from external programmer 24 via telemetry interface 70. Hence, external programmer 24 may program IMD 16 with different sets of operating parameters. In some examples, stimulation generator 66 may include a switch matrix. Processor 60 may control the switch matrix to selectively deliver stimulation pulses from stimulation generator 66 to different electrodes 29 carried by lead 28 (FIG. 1). In some examples, processor 60 may control stimulation generator to deliver electrical stimulation including a series of pulse consistent with one or more example waveforms described herein. In some examples, IMD 16 may deliver different stimulation programs to patient 14 on a time-interleaved basis with one another.

Processor 60 may access a clock or other timing device 64 within IMD 16 to determine pertinent times, e.g., for enforcement of therapy schedules, lockout periods, and therapy windows, and may synchronize such times with times maintained by external programmer 24. In various examples, IMD 16 may include one or more processors, including processor 60, such as one or more microprocessors, digital signal processors (DSPs), application specific integrated circuits (ASICs), field programmable gate arrays (FPGAs), or any other equivalent integrated or discrete logic circuitry, as well as any combinations of such components.

Memory 62 stores instructions for execution by processor 60, including operational commands and programmable parameter settings. Example storage areas of memory 62 may include instructions associated with one or more therapy programs, which may include each program used by IMD 16 to define parameters and electrode combinations for gastric stimulation therapy. Memory 62 may store one or more therapy programs containing instructions for delivering a series of electrical stimulation pulses consistent with one or more example waveform described herein. Memory 62 may include one or more memory modules constructed, e.g., as random access memory (RAM), read-only memory (ROM), non-volatile random access memory (NVRAM), electrically erasable programmable read-only memory (EEPROM), and/or FLASH memory. Processor 60 may access memory 62 to retrieve instructions for control of stimulation generator 66 and telemetry interface 70, and may store information in memory 62, such as operational information.

To facilitate delivery of stimulation in a closed loop manner, a stimulation therapy programs stored on memory 62 and executed by processor 60 may include, in one example of treating dysfunction of bladder 12 of patient 14, a baseline contraction frequency or a threshold contraction frequency. The baseline contraction frequency may be contraction frequency of bladder 12 at a time prior to delivery of stimulation therapy by stimulation generator 66. For example, the baseline contraction frequency of bladder 12 may be sensed and determined by processor 60 after implantation of IMD 16 in patient 14, but before the processor controls stimulation generator 66 to deliver any stimulation therapy to patient 14. In some examples, the baseline contraction frequency of bladder 12 may represent the patient state when no therapeutic effects from delivery of stimulation by IMD 16 are present.

Processor 60 may determine the baseline contraction frequency of bladder 12 utilizing signals representative of physiological parameters received from at least one of sensor 22, electrodes 19 or electrodes 21. In some examples, processor 60 monitors impedance of bladder 12 to detect contraction of bladder 12 based on signals received from impedance module 68. For example, processor 60 may determine an impedance value based on signals received from impedance module 68 and compare the determined impedance value to a threshold impedance value stored in memory 62. Processor may detect a contraction of bladder 12 when the processor determines an impedance value from impedance module 68 is less than the threshold value stored in memory 62. In some implementations, processor 60 monitors impedance of bladder 12 thr a predetermined duration of time to detect contractions of bladder 12, and determines the baseline contraction frequency of bladder 12 by determining a number of contractions of bladder 12 in the predetermined duration of time.

In other examples, processor 60 may monitor signals received from sensor 22 to detect contraction of bladder 12 and determine the baseline contraction frequency. In some examples, sensor 22 may be a pressure sensor for detecting changes in pressure of bladder 12, which processor 60 may correlate to contractions of bladder 12. Processor 60 may determine a pressure value based on signals received from sensor 22 and compare the determined pressure value to a threshold value stored in memory 62 to determine whether the signal is indicative of a contraction of bladder 12. In some implementations, processor 60 monitors pressure of bladder 12 to detect contractions of bladder 12 for a predetermined duration of time, and determines a contraction frequency of bladder 12 by calculating a number of contractions of bladder 12 in the predetermined time period.

Wireless telemetry in IMD 16 may be accomplished by radio frequency (RE) communication or proximal inductive interaction of IMD 16 with external programmer 24 via telemetry interface 70. Processor 60 controls telemetry interface 70 to exchange information with external programmer 24. Processor 60 may transmit operational information and receive stimulation parameter adjustments or parameter sets via telemetry interface 70. Also, in some examples, IMD 16 may communicate with other implanted devices, such as stimulators or sensors, via telemetry interface 70. In some examples, telemetry interface 70 may be configured to wirelessly communicate with other devices using non-inductive telemetry.

Power source 72 delivers operating power to the components of IMD 16. Power source 72 may include a battery and a power generation circuit to produce the operating power. In some examples, the battery may be rechargeable to allow extended operation. Recharging may be accomplished through proximal inductive interaction between an external charger and an inductive charging coil within IMD 16. In other examples, an external inductive power supply may transcutaneously power IMD 16 whenever stimulation therapy is to occur.

IMD 16 is coupled to electrodes 29 via lead 28 shown in FIG. 1. IMD 16 provides stimulation therapy to treat dysfunction of bladder 14 of patient 14. Stimulation generator 66 includes suitable signal generation circuitry for generating a voltage or current waveform with a selected amplitude, pulse width, pulse rate, and duty cycle. As described in this disclosure, the series of electrical stimulation pulses generated by stimulation generator 66 may be formulated with particular parameter values to define a waveform that is suitable to deliver stimulation from an origination site to a remote delivery site within patient 14. For example, as will be described in further detail below, IMD 16 may deliver stimulation to patient 14 defined by a series of charge balanced electrical stimulation pulses that are modulated by a high frequency carrier signal.

In one example, processor 60 controls stimulation generator 66 to delivery stimulation therapy to patient 14 according to one or more programs stored in memory 62. The program or programs by which processor 60 controls stimulation generator 66 may define stimulation waveforms characterized by parameters and values thereof described in examples according to this disclosure. For example, a stimulation program employed by processor 60 to control stimulation generator 66 may define a waveform designed to penetrate tissue within patient 14 to transmit electrical stimulation from an origination site to remote delivery site within the patient. In one example, processor 60 controls stimulation generator 66 according to a program that defines a waveform that includes successive electrical pulses of alternating polarity, each of which is modulated by a high frequency carrier signal and substantially charge balanced with preceding and subsequent pulses.

In one example, processor 60 employs a program stored on memory 62 to deliver a series of electrical pulses including a first frequency and alternating pulse polarities from an origination site to a remote delivery site within patient 14. The series of electrical pulses defined in the program and employed by processor 60 to control stimulation generator 66 may be modulated with a carrier signal including a second frequency such that each pulse in the series of electrical pulses includes a series of carrier pulses. The program employed by processor 60 may include one or more values for the first frequency that define the stimulation waveform to produce one of a number of particular therapeutic effects, e.g. to modulate contraction of bladder 14. In some examples. IMD 16 may store in memory 62 a number of programs or groups of programs defining values or ranges of values for the first frequency that define one or more stimulation waveforms delivered by stimulation generator 66 to produce a number of different therapeutic effects.

For example, a program or group of programs stored in memory 62 and employed by processor 60 to control stimulation generator 66 may include a value or range of values for the first frequency of the series of electrical pulses that is designed to treat a urinary tract dysfunction of patient 14, including, e.g. incontinence. In another example, a program or group of programs stored in memory 62 and employed by processor 60 to control stimulation generator 66, in addition to or in lieu of the urinary tract programs, may include a value or range of values for the first frequency of the series of electrical pulses that is designed to treat pain experienced by patient 14 via peripheral nerve stimulation (PNS).

In one example, processor 60 employs a program stored in memory 62 to control stimulation generator 66 to generate and deliver a series of alternating polarity electrical pulses including a first frequency in a range from approximately 4 hertz (Hz) to approximately 100 Hz from an origination site to a remote delivery site within patient 14. In one example, processor 60 employs a program stored in memory 62 to control stimulation generator 66 to generate and deliver a series of alternating polarity electrical pulses including a first frequency in a range from approximately 5 Hz to approximately 25 Hz from an origination site to a remote delivery pelvic floor stimulation site within patient 14. In one example, processor 60 employs a program stored in memory 62 to control stimulation generator 66 to generate and deliver a series of alternating polarity electrical pulses including a first frequency in a range from approximately 5 Hz to approximately 14 Hz from an origination site to a remote delivery pelvic floor stimulation site within patient 14. In one example, processor 60 employs a program stored in memory 62 to control stimulation generator 66 to generate and deliver a series of alternating polarity electrical pulses including a first frequency approximately equal to 80 Hz from an origination site to a remote delivery peripheral nerve stimulation site within patient 14.

The program employed by processor 60 may also include one or more values for the second frequency of the carrier signal to enable the series of electrical pulses delivered by stimulation generator 66 to penetrate tissue of patient 14 between an origination site and a remote delivery site. In some examples, IMD 16 may store in memory 62 a number of programs or groups of programs defining values or ranges of values for the second frequency that define one or more stimulation waveforms delivered by stimulation generator 66, which may vary based on the effectiveness of a selected second frequency of the carrier signal to penetrate patient tissue in a particular stimulation application, e.g. pelvic floor versus peripheral nerve stimulation, and/or patient anatomy. In one example, processor 60 employs a program stored in memory 62 to control stimulation generator 66 to generate and deliver a series of electrical pulses modulated by a carrier signal including a second frequency in a range from approximately 1 kilohertz (kHz) to approximately 500 kHz. In one example, processor 60 employs a program stored in memory 62 to control stimulation generator 66 to generate and deliver a series of electrical pulses modulated by a carrier signal including a second frequency in a range from approximately 4 kilohertz (kHz) to approximately 400 kHz. In one example, processor 60 employs a program stored in memory 62 to control stimulation generator 66 to generate and deliver a series of electrical pulses modulated by a carrier signal including a second frequency approximately equal to 200 kHz.

The program or programs by which processor 60 functions to control stimulation generator 66 to delivery stimulation to patient 14 may include values of a number of stimulation parameters, including, e.g. pulse width and amplitude. In one example, stimulation generator 66 may deliver stimulation to patient 14 via a series of electrical pulses with a controlled substantially constant voltage. In such examples, processor 60 functions to control stimulation generator 66 to deliver stimulation to patient 14 via a series of charge balanced electrical pulses modulated by a high frequency carrier signal employing a program that includes voltage amplitude values for the electrical pulses that are greater than zero but less than or equal to approximately 25 volts. In one example, stimulation generator 66 may deliver stimulation to patient 14 via a series of electrical pulses with a controlled substantially constant current. In such examples, processor 60 functions to control stimulation generator 66 to deliver stimulation to patient 14 via a series of charge balanced electrical pulses modulated by a high frequency carrier signal employing a program that includes current amplitude values for the electrical pulses that are greater than zero but less than or equal to approximately 25 milliamps.

The program or programs by which processor 60 functions to control stimulation generator 66 to delivery stimulation to patient 14 may include pulse width values for one or both of the series of electrical pulses and the carrier pulses of the high frequency carrier signal modulating the electrical pulses. The pulse width of the carrier pulses may be, in some examples, be set as a function of the frequency of the carrier signal. For example, a carrier signal with a frequency of 100 kHz may include pulses with pulse widths in a range from greater than zero but less than or equal to approximately 1/100,000 seconds or 10 microseconds (μs). In another example, a carrier signal with a frequency of 200 kHz may include pulses with pulse widths in a range from greater than zero but less than or equal to approximately 1/200,000 seconds or 200 milliseconds (μs).

The program or programs by which processor 60 functions to control stimulation generator 66 to delivery stimulation to patient 14 may also include pulse width values for the series of electrical pulses, in one example, processor 60 controls stimulation generator 66 to deliver a series of electrical pulses to patient 14 with a pulse width in a range from approximately 100 μs to approximately 5 ms. In another example, processor 60 controls stimulation generator 66 to deliver a series of electrical pulses to patient 14 with a pulse width in a range from approximately 200 μs to approximately 1 ins.

In the example of FIGS. 1 and 3, IMD 16 includes leads 18, 20, and 28. As noted above, however, in other examples, IMD 16 may be a leadless stimulator, sometimes referred to as a microstimulator, or combination of such stimulators. In this case, the housing of IMD 16 may include multiple electrodes to form electrode combinations for delivery of stimulation to the pelvic floor nerves, peripheral nerves, the spinal cord, or other locations within patient 14. In additional examples, IMD 16 may include more or fewer leads than the examples of FIGS. 1 and 3.

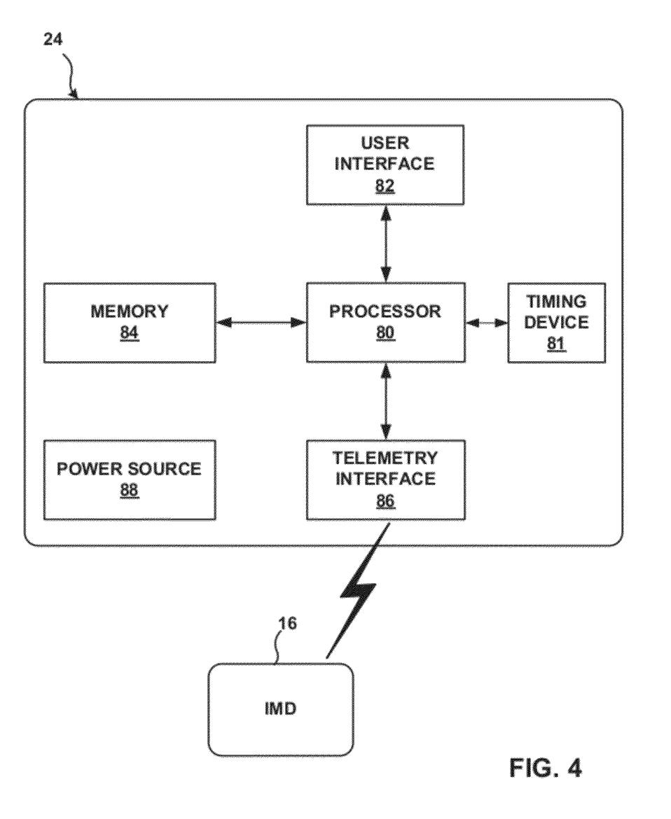

FIG. 4 is a block diagram illustrating example components of external programmer 24 that receives user input and communicates with IMD 16. External programmer may be employed by patient 14 or another user, e.g. a clinician to control the stimulation therapy delivered by IMD 16. External programmer 24 includes processor 80, user interface 82, memory 84, telemetry interface 86 and power source 88. In addition, processor 80 may access a clock or other timing device 81 to adhere to lockout periods, therapy windows, and therapy schedules, as applicable. In some examples, patient 14 may carry external programmer 24 throughout therapy so that the patient can initiate, stop and/or adjust stimulation as needed.

While external programmer 24 may be any type of computing device, the external programmer may preferably be a hand-held device with a display and input mechanism associated with user interface 82 to allow interaction between, e.g., patient 14 and external programmer 24. In some examples, external programmer 24 may be employed by a clinician to program IMD 16. In such examples, user interface 82 of external programmer 24 may include additional features not offered to patient 14 for security, performance, or complexity reasons.

User interface 82 may include display and keypad (not shown), and may also include a touch screen or peripheral pointing devices. User interface 82 may be designed to receive an indication from patient 14 to deliver stimulation therapy via IMD 16. The indication may be in the form of a patient input in the form of pressing a button representing the start of therapy or selecting an icon from a touch screen, for example. In alternative examples, user interface 82 may receive an audio cue from patient 14, e.g., the patient speaks to a microphone in order to perform functions such as beginning stimulation therapy. External programmer 24 acts as an intermediary for patient 14 to communicate with IMD 16 thr the duration of therapy.

User interface 82 may provide patient 14 with information pertaining, for example, to the status of an indication or a stimulation function. Upon receiving the indication to start stimulation, user interface 82 may present a confirmation message to patient 14 that indicates stimulation has begun. The confirmation message may be a picture, icon, text message, sound, vibration, or other indication that communicates the therapy status to patient 14.

In examples in which programmer 24 is employed by a clinician, the clinician may use the programmer to modify and review stimulation therapy delivered to patient 14. With the aid of programmer 24, the clinician may define each therapy parameter value for each of the programs that define stimulation therapy and program IMD 16 with the selected therapy parameter values. The therapy parameter values, such as pulse width and frequency, may be defined specifically for each of the series of electrical pulses and the carrier signal by which the series of the electrical pulses is modulated.

Processor 80 may include one or more processors such as a microprocessor, a controller, a DSP, an ASIC, an FPGA, discrete logic circuitry, or the like, Processor 80 may control information displayed on user interface 82 and perform certain functions when requested by patient 14 or another user via input to the user interface. Processor 80 may retrieve data from and/or store data in memory 84 in order to perform the functions of external programmer 24 described herein. For example, processor 80 may generate a series of electrical stimulation pulses consistent with one or more example waveforms described herein based upon instructions stored in memory 84, and processor 80 may then store the selection in memory 84.

Memory 84 may store information relating to the one or more stimulation programs used to define therapy delivered to patient 14. When a new program is requested by IMD 16 or patient 14 or another user, parameter information corresponding to one or more of the therapy programs may be retrieved from memory 84 and transmitted to 16 in order adjust the stimulation therapy. Alternatively, patient 14 may generate a new program during therapy and store it within memory 84. Memory 84 may include any volatile, non-volatile, fixed, removable, magnetic, optical, or electrical media, such as a RAM, ROM, CD-ROM, hard disk, removable magnetic disk, memory cards or sticks, NVRAM. EEPROM, flash memory, and the like.

Processor 80 in conjunction with memory 84 may be employed to perform some or all of the functions described with reference to processor 60 and memory 62 of MID 16. For example, processor 80 may control stimulation generator 66 of IMD 16 by transmitting stimulation parameter values or entire programs to the device via telemetry interface 86 to set and/or adjust stimulation parameters such as pulse amplitude, pulse rate, pulse width and frequency.

Telemetry interface 86 allows the transfer of data to and from IMD 16. Telemetry interface 86 may communicate automatically with IMD 16 at a scheduled time or when the telemetry interface detects the proximity of the IMD. Alternatively, telemetry interface 86 may communicate with IMD 16 when signaled by a user through user interface 82. To support RF communication, telemetry interface 86 may include appropriate electronic components, such as amplifiers, filters, mixers, encoders, decoders, and the like.

Power source 88 may be a rechargeable battery; such as a lithium ion or nickel metal hydride battery. Other rechargeable or conventional batteries may also be used. In some cases, external programmer 24 may be used when coupled to an alternating current (AC) outlet, i.e., AC line power, either directly or via an AC/DC adapter.

In some examples, external programmer 24 may be configured to recharge IMD 16 in addition to programming the MD. Alternatively; a recharging device may be capable of communication with IMD 16. In one such example, the recharging device may be able to transfer programming information, data, or any other information described herein to IMD 16. In this manner, the recharging device may be able to act as an intermediary communication device between external programmer 24 and IMD 16. Generally speaking, the techniques for delivering stimulation to patient 14 via high frequency modulated and charge balanced electrical stimulation waveforms described in this disclosure may be communicated to and from IMD 16 via any type of external device capable of electronic communications therewith.

While external programmer 24 is generally described as a hand-held computing device, the external programmer may be a notebook computer, a cell phone, or a workstation, for example. In some examples, external programmer 24 may comprise two or more separate devices that perform the functions ascribed to the external programmer. In one example in which programmer 24 is employed as a patient programmer, patient 14 may carry a key fob that is only used to start or stop stimulation therapy. The key fob may then be connected to a larger computing device having a screen via a wired or wireless connection when information between the two needs to be synchronized. Alternatively, external programmer 24 may simply be small device having one button, e.g., a single “start” button, that only allows patient 14 to start stimulation therapy when the patient feels hungry or is about to eat.

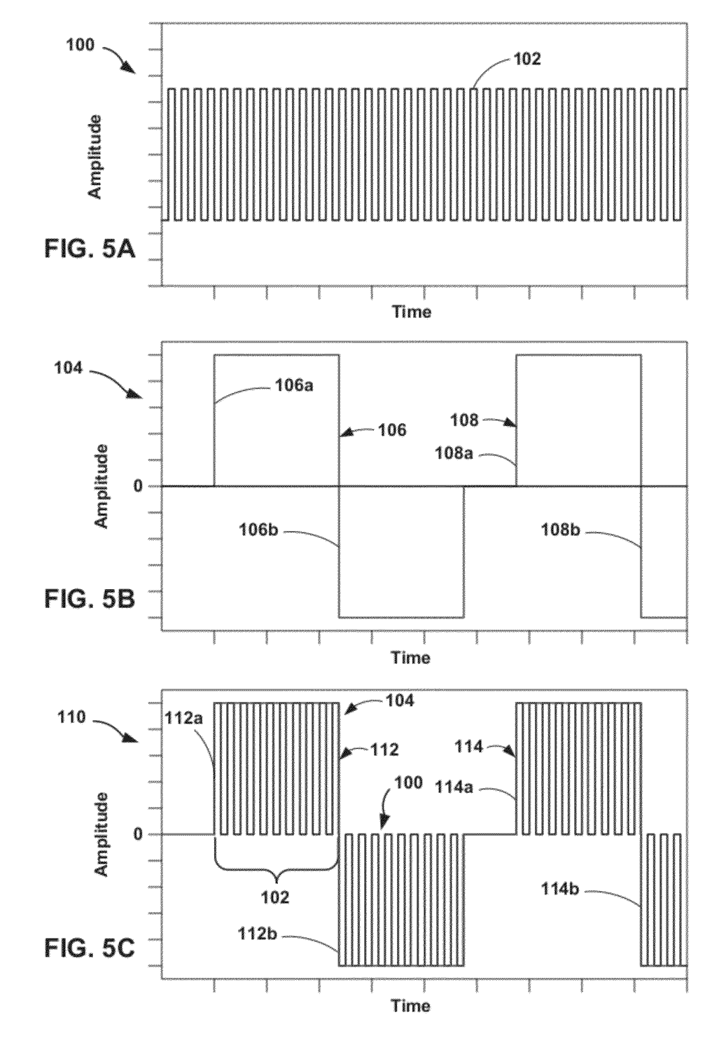

As described above, according to some examples of the disclosure, a medical device, such as, e.g., IMD 16 (FIG. 1), may be configured to generate and deliver electrical stimulation therapy to patient 14. The electrical stimulation therapy generated and delivered to patient from the medical device may include a series of electrical pulses modulated by a high frequency carrier signal and charge balanced that are consistent with one or more example waveforms described herein. FIGS. 5A-8 illustrate example waveforms defined by a series of electrical pulses, which may be generated and delivered to a patient by a medical device, e.g., to treat one or more disorders or diseases, e.g. a urinary tract dysfunction. For purposes of illustration, the example waveforms of FIGS. 5A-8 are described with regard to therapy system 10 of FIG. 1. However, examples of the disclosure may be incorporated into any suitable medical system or device capable of delivering electrical stimulation to a patient.

In some examples, two successive electrical stimulation pulses can be characterized as being coupled to one another. A coupled pair of electrical stimulation pulses may include a first electrical stimulation pulse of one polarity (anodic or cathodic) followed immediately, or with some fixed delay, by second electrical stimulation pulse of opposite polarity. When the coupled pair of electrical stimulation pulses are charge balanced, the charge of the first pulse is equal to but opposite of that of the charge of the second pulse. Notably, unlike uncoupled pulses, the timing of the delivery of two stimulus pulses that are coupled to one another is fixed. For example, for a plurality of pulses including multiple coupled pairs of pulses in which each coupled pair includes a first anodic pulse followed by a second cathodic pulse that are charged balanced, each of the coupled pairs of pulses may be delivered relative to each other at a set frequency that may be varied. However, the temporal relationship of the each pulses in a pair of coupled pulses is unaffected by the chosen frequency for delivery of the each coupled pair of pulses relative to one another. For example, while the interval between the leading edges of successive pulses of the same polarity will be longer at lower selected pulse frequencies, and shorter at higher selected pulse frequencies, the temporal relationship of the pair of coupled pulses of each coupled pair is unaffected by the chosen pulse frequency. The time elapsed between the first pulse and the second pulse is fixed, (e.g., at approximately zero or some fixed time delay) regardless of the selected frequency at which the coupled pairs of pulses are delivered.

FIGS. 5A-5C illustrate an example waveform defined by a series of charge balanced electrical pulses modulated by a high frequency carrier signal. FIG. 5A illustrates an example high frequency carrier signal that may be employed in examples according to this disclosure to modulate a series of electrical pulses delivered to a patient. FIG. 5B illustrates an example series of charge balanced electrical pulses configured to be delivered to a patient to stimulate tissue and thereby provide therapy for one or more conditions of the patient. FIG. 5C illustrates the series of charge balanced electrical pulses of FIG. 5B modulated by the high frequency carrier signal of FIG. 5A.

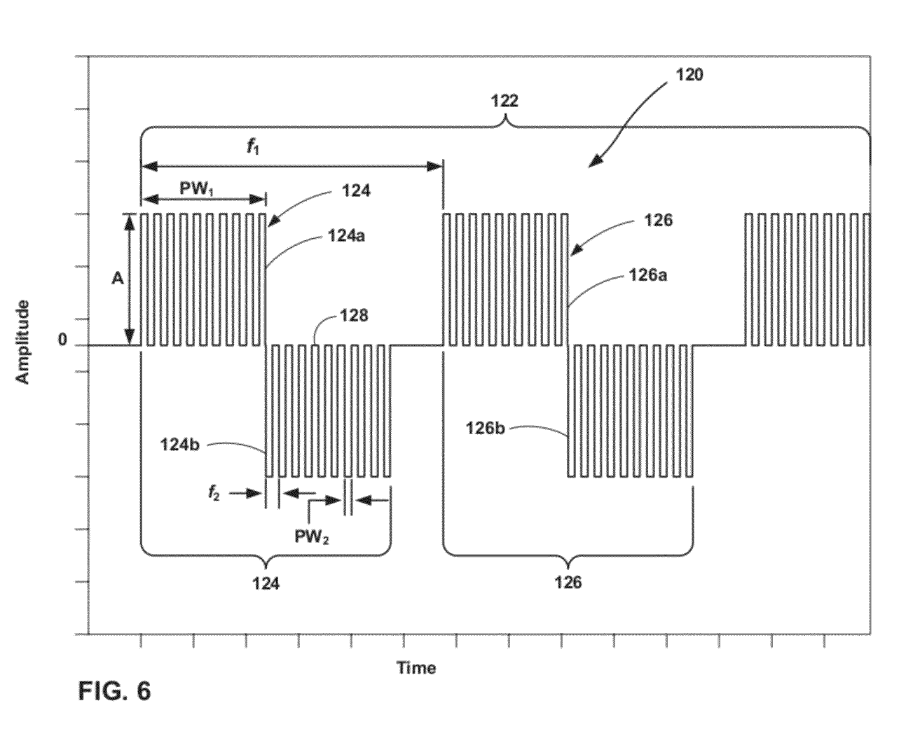

FIG. 5A illustrates example high frequency carrier signal 100 that may be employed in examples according to this disclosure to modulate a series of electrical pulses delivered to patient 14 by IMD 16. In FIG. 5A, carrier signal 100 includes a series of carrier pulses 102. As described above and below in greater detail with reference to FIG. 6, carrier signal 100 may be characterized by relatively high frequencies relative to the frequency of a series of electrical pulses the carrier signal modulates. For example, carrier signal 100 may include a frequency in a range from approximately 1 kHz to approximately 500 kHz, compared to a frequency of a series of electrical pulses in a range from approximately 4 Hz to approximately 100 Hz.

FIG. 5B illustrates example waveform 104 including a series of charge balanced electrical pulses configured to be delivered to patient 14 by IMD 16 to stimulate tissue and thereby provide therapy for one or more conditions of the patient. In FIG. 5B, waveform 104 includes a series of two pulses 106 and 108. In the example of FIG. 5B, pulses 106 and 108 are biphasic pulses, each of which includes coupled pairs of pulses 106a, 106b and 108a, 108b, respectively. Each of coupled pairs of pulses 106a, 106b and 108a, 108b are charge balanced such that the net charge of each coupled pair of pulses is approximately equal to zero. For example, biphasic pulse 106 includes coupled pair of pulses 106a, 106b, which include approximately equal charges of opposite polarity. As such, the net charge of coupled pair of pulses 106a, 106b is approximately equal to zero, in examples including biphasic pulses, each of which includes a coupled pair of pulses, the two pulses in a biphasic pulse may also be referred to as the two phases of the biphasic pulse. For example, biphasic pulse 106 may be referred to as including first phase 106a and second phase 106b.

FIG. 5C illustrates series of charge balanced electrical pulses 104 of FIG. 5B modulated by high frequency carrier signal 100 of FIG. 5A to define example waveform 110 by which IMD 16 may deliver electrical stimulation therapy to patient 14. In FIG. 5C, waveform 110 includes a series of electrical pulses 104. The series of electrical pulses 104 includes a first frequency and alternating pulse polarities. The series of electrical pulses 104 is modulated by carrier signal 100 including a second frequency such that each pulse in the series of electrical pulses, e.g. 112a, 112b, 114b includes a series of carrier pulses 102 including a single polarity and the second frequency of the carrier signal. In the example of FIG. 5C, example waveform 110 is defined by two biphasic pulses 112 and 114. Each of biphasic pulses 112., 114 includes a coupled pair of pulses, i.e. coupled pair 112a, 112b and 114a, 114b, respectively. Each pair of successive pulses in the series of electrical pulses 104 is substantially charge balance. For example, biphasic pulse 112. Includes coupled pair of pulses 112a, 112b, which include approximately equal charges of opposite polarity. As such, the net charge of coupled pair of pulses 112a, 112b is approximately equal to zero. The configuration and characteristics of a series of charge balanced electrical pulses modulated by a high frequency carrier signal as illustrated in FIGS. 5A-5C is described in greater detail with reference to another example waveform in FIG. 6.

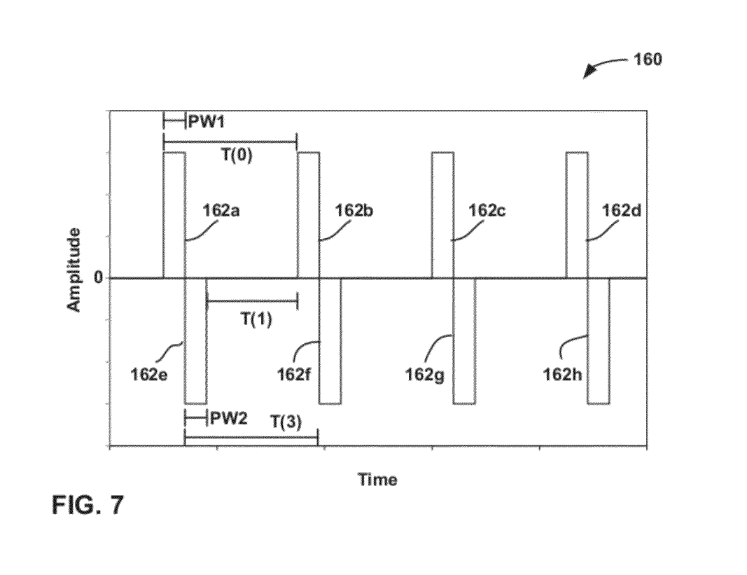

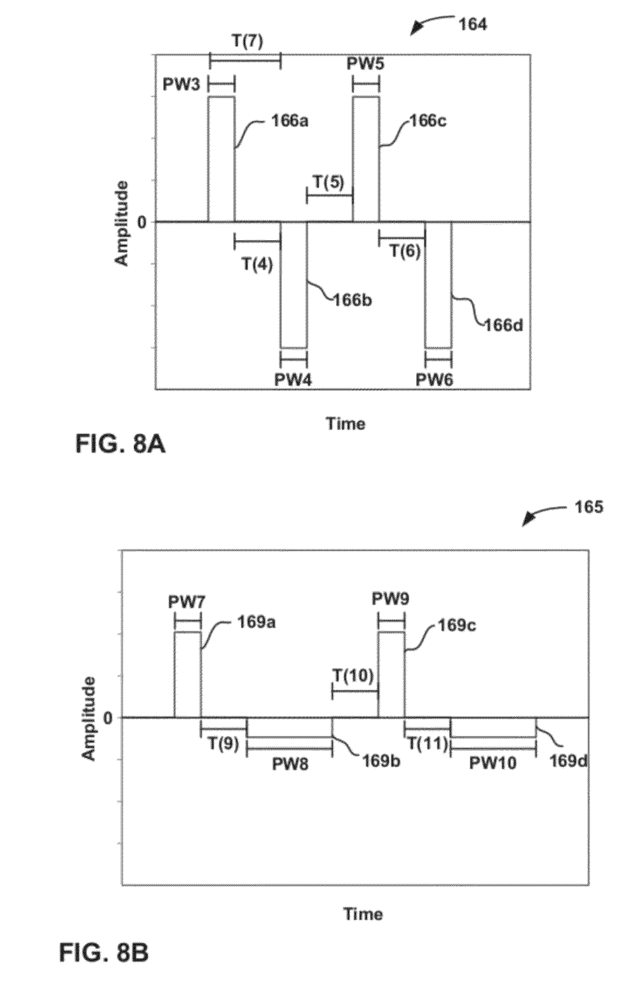

FIG. 6 illustrates example waveform 120 by which IMD 16 may deliver electrical stimulation therapy to patient 14. In FIG. 6, waveform 120 is defined by a series of electrical stimulation pulses 122, including a number of biphasic pulses, e.g. biphasic pulses 124 and 126. The series of electrical pulses 122 includes a first frequency, f₁, and successive pulses of alternating polarity. In the example of FIG. 6, the series of electrical pulses 122 includes a number of biphasic pulses, including biphasic pulses 124 and 126. Each of biphasic pulses 124, 126 includes a coupled pair of pulses, i.e. 124a, 124b and 126a, 126b, respectively. Biphasic pulses are described in greater detail with reference to FIG. 7 below. In other examples according to this disclosure, e.g. as illustrated below with reference to FIG. 8A, a series of electrical pulses used to deliver stimulation therapy to patient 14 may take other forms, e.g. a series of monophasic pulses of opposite polarity.

The first frequency, f₁, of the series of electrical pulses 122 is defined by the rate at which each successive biphasic pulse in the series is delivered. For example, in FIG. 6, the first frequency, of the series of electrical pulses 122 is equal to one divided by the period of time between the beginning of biphasic pulse 124 and the beginning of biphasic pulse 126. The first frequency, f₁, of the series of electrical pulses 122 may include a range of values, which may be configured to produce a particular therapeutic effect. For example, the first frequency, f₁, of the series of electrical pulses 122 may be in a range from approximately 4 hertz (Hz) to approximately 100 Hz. In one example, the first frequency, f₁, of the series of electrical pulses 122 may be configured to treat a urinary tract dysfunction, e.g., stress incontinence or urge incontinence of patient 14 and may be in a range from approximately 5 Hz to approximately 25 Hz. In another example, the first frequency, f₁, of the series of electrical pulses 122 may be configured to treat a urinary tract dysfunction of patient 14 and may be in a range from approximately 5 Hz to approximately 14 Hz. In another example, the first frequency, f₁, of the series of electrical pulses 122 may be configured to treat a pain experienced by patient 14 via peripheral nerve stimulation and may be approximately equal to 80 Hz.

Each pulse in series 122 in the example of FIG. 6 is also defined by an amplitude, A, and a pulse width PW₁. In one example, waveform 120 is delivered by IMD 16 via a controlled substantially constant voltage source and the amplitude of each pulse in the series of electrical pulses 122 may include an amplitude in a range from greater than zero to approximately 25 volts (V). In one example, waveform 120 is delivered by IMD 16 via a controlled substantially constant current source and the amplitude of each pulse in series 122 may include an amplitude in a range from greater than zero to approximately 25 milliamps (mA).

The pulse width, PW_{I}, of each pulse in the series of electrical pulses 122 in the example of FIG. 6, in one example, may be in a range from approximately 100 μs to approximately 5 ms. In another example, the pulse width, PW₁, of each pulse in series 122 in the example of FIG. 6, in one example, may be in a range from approximately 200 μs to approximately 1 ms.

The series of electrical pulses 122 defining waveform 120 in FIG. 6 is modulated by a high frequency carrier signal such that each pulse in the series of electrical pulses, e.g. each of pulse 124a, 124b, 126a, 126b includes a series of carrier pulses 128 including a single polarity and a second frequency, f₂. The second frequency, f₂, of the carrier signal including carrier pulses 128 may be configured to enable the series of electrical pulses 122 to penetrate tissue between an origination site from which IMD 16 delivers the pulses and a remote delivery site within patient 14. In one example, the second frequency, f₂, of the carrier signal including carrier pulses 128 may be in a range from approximately 1 kHz to approximately 500 kHz. In another example, the second frequency, f₂, of the carrier signal including carrier pulses 128 may be in a range from approximately 4 kHz to approximately 400 kHz. In another example, the second frequency, f₂, of the carrier signal including carrier pulses 128 may be approximately equal to 200 kHz.

Each carrier pulse 128 included in each electrical pulse in series 122 may also be defined by a pulse width, PW₂. In one example, the pulse width, PW₂, of one or more of carrier pulses 128 may be defined as a function of the second frequency, f₂, of the carrier signal. For example, the pulse width, PW₂, of one or more of carrier pulses 128 may be in a range from greater than zero but less than or equal to approximately one divided by the second frequency, f₂, of the carrier signal. For example, a carrier signal with a frequency of 100 kHz may include carrier pulses 128 with pulse widths, PW₂, in a range from greater than zero but less than or equal to approximately 1/100,000 seconds or 10 microseconds (μs). In another example, a carrier signal with a frequency of 200 kHz may include carrier pulses 128 with pulse widths, PW₂, in a range from greater than zero but less than or equal to approximately 1/200,000 seconds or 200 milliseconds (μs).

Each pair of successive pulses in the series of electrical pulses 12 is substantially charge balanced. For example, biphasic pulse 124 includes coupled pair of pulses 124a, 124b, which include approximately equal charges of opposite polarity. As such, the net charge of coupled pair of pulses 124a, 124b is approximately equal to zero. Similarly, biphasic pulse 126 includes coupled pair of pulses 126a, 126b, which include approximately equal charges of opposite polarity. As such, the net charge of coupled pair of pulses 126a, 126b is approximately equal to zero.

In the example of FIG. 6, each of the electrical pulses modulated by the high frequency carrier signal in the series 122 is substantially the same. For example, pulse 124a and pulse 124b in biphasic pulse 124 are defined such that pulse 124b includes a polarity opposite the polarity of the pulse 124b and a pulse width, amplitude, and carrier signal frequency approximately equal to the pulse width, amplitude, and carrier signal frequency of pulse 124a. Furthermore, in the example of FIG. 6, the pulse width of carrier pulses 128 included in each of pulses 124a and 124b are equal and are constant throughout the duration of each of pulses 124a and 124b. In some examples, successive pulses in the series of electrical pulses 122 may remain charge balanced but vary in some parameters. For example, a first pulse may include a first amplitude and a first pulse width and a second pulse may include a second amplitude that his less than the first amplitude and a second pulse width that is greater than the first pulse width such that, despite the differences in amplitudes and pulse widths, the first and second successive pulses are nevertheless substantially charge balanced. In the case of charge balanced biphasic pulses, this type of varying amplitude a pulse width setting between the pair of coupled pulses in a biphasic pulse may produce what is referred to as an asymmetric biphasic pulse. Additionally, in some examples, the parameters of carrier pulses 128 may vary in the series of electrical pulses 122. In one example, the pulse width of carrier pulses 128 may vary within a single electrical pulse in the series of electrical pulses 122. In such an example, the pulse width variance in carrier pulses 128 within a single electrical pulse may be matched in a successive electrical pulse such that the two successive pulses are substantially the same, but of opposite polarity.

FIGS. 7-10 illustrate a number of forms a series of electrical pulses used to deliver stimulation therapy to patient 14 may take in examples according to this disclosure, including biphasic in FIGS. 7, 9, and 10 and monophasic in FIGS. 8A and 8B. In the examples of FIGS. 7-10 the high frequency carrier signal is omitted, as the examples are intended to illustrate features of electrical pulses, which are modulated by the high frequency carrier signal in examples according to this disclosure.

FIG. 7 is a plot illustrating an example waveform 160 representing an example series of electrical stimulation pulses for delivery to patient 14. In particular, in FIG. 7, waveform 160 includes first stimulation pulse 162a, second stimulation pulse 162b, third stimulation pulse 162c, fourth stimulation pulse 162d, fifth stimulation pulse 162e, sixth stimulation pulse 162f, seventh stimulation pulse 162g, and eighth stimulation pulse 162h (collectively “series of stimulation pulses 162”). IMD 16 may generate and deliver electric stimulation to patient 14 via electrodes 29 carried on lead 28, where the electric stimulation includes the series of electrical stimulation pulses 162 represented by waveform 160. In some examples, such electric stimulation may treat one or more conditions of patient 14. Although the series of stimulation pulses 162 represented by waveform 160 are shown to include stimulation pulses 162a-162h, the electric stimulation generated and delivered to patient 14 by IMD 16 may include any number of stimulation pulses that provide effective treatment to patient 14.

As shown in FIG. 7, the series of stimulation pulses 162 is a plurality of pulses including pairs of individual pulses that are coupled to one another. In particular, first pulse 162a is coupled with fifth pulse 162e, second pulse 162b is coupled with sixth pulse 162f, third pulse 162c is coupled with seventh pulse 162g, and fourth pulse 162d is coupled with eighth pulse 162h. As such, the temporal relationship between each pulse of a coupled pair of pulses is fixed. In the example shown in FIG. 7, the temporal relationship between each pulse of coupled pairs of pulses (e.g., pulses 162a and 162e, pulses 162b and 162f, and so forth) is such that the second pulse of the coupled pair (e.g., pulses 162e, 162f, 162g, and 162h) is delivered substantially immediately after the first pulse of the coupled pair (e.g., pulses 162a, 162b, 162c, and 162d, respectively) ends. The pulse width of each first pulse (i.e., pulses 162a-d) of the coupled pairs of pulses (which is equal to pulse width PW 1) is substantially equal to the pulse width of each second pulse (i.e., pulses 162e-h) of the coupled pairs of pulses (which is equal to pulse width PW2). Each first pulse (i.e., pulses 162a-d) of a coupled pair has substantially the same amplitude but opposite polarity of each second pulse (i.e., pulses 162e-h) of a coupled pair. In some examples, the fixed time delay between pulses of coupled pulse pairs may be less than the pulse width of the pulses of the pulse pair, e.g., less than pulse width PW1 of first pulse 162a.

In waveform 160, the coupled pairs of pulses (pulses 162a and 162e, pulses 162b and 162f, and so forth) are delivered at a set frequency, which is consistent with time interval T(0) As described above, the frequency that the coupled pairs of pulses are delivered does not change the timing that between each pulse in a coupled pair. After IMD 16 delivers fifth pulse 162e of patient 14, there is a delay interval T(1) prior to the beginning of the subsequent coupled pulse pair (pulses 162b and 162f). Unlike that of the fixed relationship between pulses of coupled pulse pairs, the timing between the delivery of respective coupled pulse pairs varies with the set frequency. For example, if the frequency of the delivery of coupled pulse pairs increases, then time intervals T(0), T(1) and T(3) decrease. Conversely, if the frequency of the delivery of coupled pulse pairs decreases, then time intervals T(0), T(1) and T(3) increase. However, the timing between each pulse of a coupled pulse pair does not change in either case.

As represented by waveform 160, IMD 16 delivers stimulation pulses 162a-h in direct succession with one another. All of stimulation pulses 162a-d have the same polarity (all cathodic or all anodic), and all of stimulation pulses 162e-h have the same polarity, which is opposite from that of the polarity of pulses 162a-d. Furthermore, each pulse of the series of pulses 162 has approximately the same amplitude and pulse width, although the polarity of the pulses alternates as indicated in FIG. 7. As such, for the series of pulses 162., the charge of each of pulses 162a-d is approximately equal to and opposite of that of the charge each of pulses 162e-h, i.e., the area between the amplitude curve and the zero amplitude line for each of pulses 162a-162d is approximately equal to the corresponding area between the amplitude curve and zero amplitude for each of pulses 162e-h. Accordingly, each coupled pair of pulses of the series of pulse 162 is charge balanced and the entire series of pulses 162 may be considered charge balanced.

In some examples, waveform 160 may be described as “symmetric rectangular biphasic” or, simply “symmetric biphasic”. Increasing the constant frequency of coupled pair pulses in FIG. 7 would decrease T(0), T(1) and T(3), but would not change the temporal relationship between the respective pulses of each coupled pulse pair. In the example shown in FIG. 7, such a frequency change would not change the fact that the first pulse of a coupled pulse pair is followed substantially immediately by a recharge pulse. Likewise, decreasing the constant frequency of coupled pulse pairs in FIG. 7 would increase T(0), T(1) and T(3), but would leave the temporal relationship between the first pulse and second pulse of a coupled pulse pair unaltered. While in the example in FIG. 7 the time between each pulse of a coupled pulse pair is substantially zero, this interval is often fixed at some fixed positive value, generally much less than the pulse width PW1.

FIG. 8A is a plot illustrating another example waveform 164 representing an example series of electrical stimulation pulses for delivery to patient 14. In particular, first stimulation pulse 166a, second stimulation pulse 166b, third stimulation pulse 166c, and fourth stimulation pulse 166d (collectively “series of stimulation pulses” 166) are represented by waveform 164. IMD 16 may generate and deliver electric stimulation to patient 14 via electrodes 29 carried on lead 28, where the stimulation includes the series of electrical stimulation pulses 166 represented by waveform 164. In some examples, such electric stimulation may effectively treat one or more conditions of patient 14. Although series of stimulation pulses 166 represented by waveform 164 are shown to include four stimulation pulses 166a-d, the electric stimulation generated and delivered to patient 14 by IMD 16 may include any number of stimulation pulses that provide effective treatment to patient 14.

As represented by waveform 164, IMD 16 delivers first stimulation pulse 166a, second pulse 166b, third stimulation pulse 166c, and fourth stimulation pulse 166d in direct succession with one another and in the order listed. In the series of stimulation pulses 166, each pulse has a polarity that is opposite of the polarity of the directly preceding pulse and the directly following pulse. For example, as delivered by IMD 16, first stimulation pulse 166a has a first polarity, which may be either anodic or cathodic, second stimulation pulse 166b has polarity opposite from that of first pulse 166a, third stimulation pulse 166c has a polarity opposite from that of second stimulation pulse 166b, and so forth.

Unlike waveform 160 (FIG. 7), in waveform 164 (FIG. 8A), a time interval that is greater than zero separates each respective pulse in the series of pulses 166. For example, a time interval T(4) greater than zero separates the trailing edge of first pulse 166a and leading edge of second pulse 166b. Similarly, a time interval T(5) greater than zero separates the trailing edge of second pulse 166b and leading edge of third pulse 166c.

Furthermore, unlike the series of pulses 162 represented by waveform 160 (FIG. 7), pulses 166a-166d do not form coupled pulse pairs with one another. Instead, the temporal relationship between each individual pulse in the series of pulses is dependent on the stimulation pulse frequency in particular, time intervals T(4), T(5) and T(6) depend on the frequency that the series of pulses are delivered and the pulse width of each pulse. If series of pulses 166 are delivered at an increased frequency while the pulse width is constant, then time intervals T(4), T(5) and T(6) all decrease. Conversely, if series of pulses 166 are delivered at an decreased frequency while the pulse width is constant, then time intervals T(4), T(5) and T(6) all increase.

In some examples, time intervals T(4), T(5) and T(6) may be substantially equal to one another such that pulses 166a-d are evenly spaced. In other examples, time interval T(4) may be different than that of time interval T(5) and/or time interval T(6). However, in each case, time intervals T(4), T(5) and T(6) are dependent on the frequency at which the series of pulses 166 are delivered since none of pulses 166a-d form coupled pulse pairs. In examples in which T(4), T(5) and T(6) are approximately equal to one another and pulses 166a-d each have approximately the same pulse width, the pulse frequency of series of pulses 166 may be determined by the time interval between the leading edge of each pulse, e.g., time interval T(7) between first pulse 166a and second pulse 166b. In some examples, the interpulse interval between directly successive pulses is not less than the pulse width of the successive pulses. For example, time interval T(4) may be greater than or equal to PW3 and PW4.

The overall charge of the series of pulses 166 of waveform 164 may be approximately zero. The charge of each pulse is dependent on the amplitude and pulse width of each respective pulse of the series of pulses 166. In some examples, the pulse width and amplitude of each respective pulse 166a-d may be selected such that the charge of first pulse 166a may be approximately equal to and opposite of that of the charge of second pulse 166b, and the charge of third pulse 166c may be approximately equal to and opposite of that of the charge of fourth pulse 166d. In some examples, each pulse of the series of pulses 166 may have approximately the same amplitude and pulse width. In other examples, the pulse width and amplitude may differ between pulses. In any case, the series of pulse 166 may be described as charge balanced even though the first pulse 166a is not followed substantially immediately by a second pulse 166b with an equal and opposite charge, as was the case in waveform 160 (FIG. 7). Instead, second pulse 166b is delivered after time interval T(4) greater than zero after the end first pulse 166a.

In some examples, waveform 166 may be referred to as representing “alternating monophasic rectangular pulses” or simply “alternating monophasic pulses”. In the example in FIG. 8A, the pulses in the sequence may have a constant width, such that PW3, PW4, PW5, and PW6 are substantially equal, and the pulses are being issued at a constant frequency, such that the time elapsed from the leading edge of one pulse in the sequence to the leading edge of the next pulse, T(7), is constant throughout the series of pulses. In the example shown in FIG. 8A. T(7) may be, e.g., 40 milliseconds, implying a constant pulse frequency of 25 Hz. Likewise, the constant pulse frequency implies that the intervals between successive pulses, during which the amplitude of stimulation delivered to the patient is approximately zero, are also of substantially equal duration, such that T(4), T(5) and T(6) in this example are substantially equal.

Unlike the symmetric biphasic waveform 160 (FIG. 7) the interval between every successive pulses of opposite polarity in waveform 164 (FIG. 8A) may vary with the pulse frequency selected. This is because each pair of adjacent rectangular pulses with opposite polarity in waveform 164 are two uncoupled stimulus pulses, rather than some of the pulses forming coupled pulse pairs. An increase in pulse frequency for the alternating monophasic waveform 164 will cause the intervals between successive stimulus pulses of opposing polarity to decrease in duration, while a decrease in the selected pulse frequency will cause these intervals between successive pulses of opposite polarity to increase.

FIG. 8B is a plot illustrating another example waveform 165 representing an example series of electrical stimulation pulses for delivery to patient 16. In particular, first stimulation pulse 169a, second stimulation pulse 169b, third stimulation pulse 169c, and fourth stimulation pulse 169d (collectively “series of electrical pulses” 169) are represented by waveform 165. The series of pulses 169 of waveform 165 are similar to the series of pulses 166 of waveform 164 in FIG. 8A. For example, none of pulses 166a-d form coupled pulse with each other. Instead, the temporal relationship between each of 166a-d is dependent on the frequency at which the series of pulses 166 is delivered. In some examples, time intervals T(9), T(10) and T(11) may be substantially the same, and may vary based on the frequency at which the series of pulses 169 are delivered to patient 16.

However, unlike that shown in FIG. 8A, the pulse width and pulse amplitude of the series of pulses 169 is not the same for each respective pulse 169a-d. In particular, first pulse 169a and third pulse 169c have substantially the same amplitude, which is greater than the amplitude of second pulse 169b and fourth pulse 169d, which also have substantially the same amplitude. Moreover, the pulse width PW 7 of first pulse 169a and pulse width PW9 of second third pulse 169c is substantially the same and less than that of the pulse width PW8 of second pulse 169b and PW10 of fourth pulse 169d, which are also substantially the same as one another. Despite the difference in pulse widths and pulse amplitude, the pulse width and amplitude of each respective pulse may be selected such that the series of pulses 169 are substantially charge balanced. For example, first pulse 169a may have substantially the same but opposite charge from the charge of second pulse 169b.

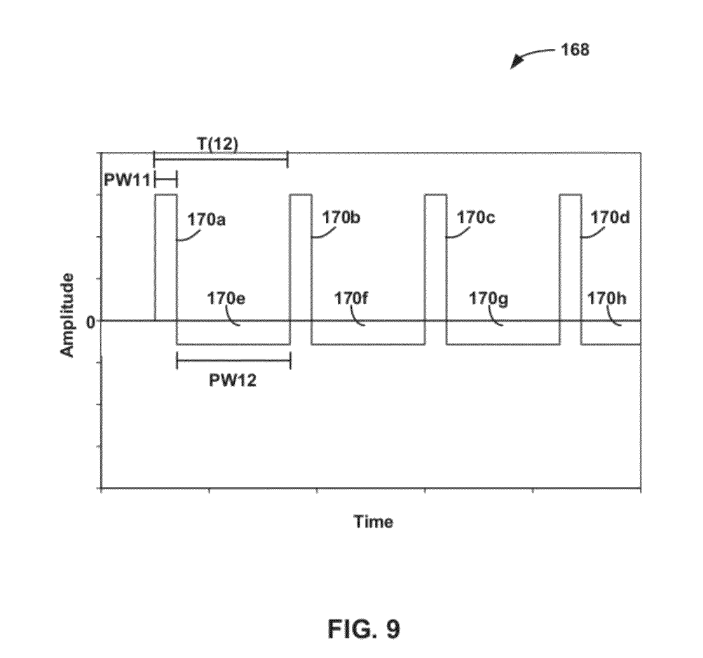

FIG. 9 is a plot illustrating another example waveform 168 representing an example series of electrical stimulation pulses for delivery to patient 16. In particular, waveform 168 includes first stimulation pulse 170a, second stimulation pulse 170b, third stimulation pulse 170c, fourth stimulation pulse 170d, fifth stimulation pulse 170e, sixth stimulation pulse 170f, seventh stimulation pulse 170g, and eighth stimulation pulse 170h (partially shown) (collectively “series of electrical pulses 170”). IMD 12 may generate and deliver electric stimulation to patient 16 via electrodes 29 carried on lead 28, where the stimulation includes the series of electrical stimulation pulses 170 represented by waveform 168. In some examples, such electric stimulation may effectively treat one or more patient conditions, e.g., a urinary tract dysfunction of patient 16. Although series of electrical pulses 170 represented by waveform 168 are shown to include eight electrical pulses 170a-h, the electric stimulation generated and delivered to patient 16 by IMD 12 may include any number of electrical pulses that provide effective treatment to patient 16.

As represented by waveform 168, IMD 12 delivers electrical pulses 170a-h in direct succession with one another. Each of electrical pulses 170a-d have the same polarity (all cathodic or all anodic), and each of electrical pulses 170e-h have the same polarity, which is opposite from that of the polarity of pulses 170a-d. As shown in FIG. 9, similar to that of waveform 160 (FIG. 7), each of pulses 170a-d is followed substantially immediately by pulses 170e-h, respectively. However, unlike waveform 160 (FIG. 6), in some examples, the pulse width of each of pulses 170e-g in waveform 168 (FIG. 9) is such that each pulse 170e-g is followed substantially immediately by pulses 170b-d, respectively. In such examples, IMD 12 delivers series of pulses 170 to patient 16 such that there is substantially no time interval between each successive pulse. Furthermore, unlike that of waveform 160 (FIG. 7), the amplitude of pulses 170a-d is different than that of the amplitude of pulses 170e-h. In particular, as shown in FIG. 9, the amplitude of pulses 170a-d is greater than that of pulses 170e-h. Additionally, the pulse width of pulses 170a-d is less than that of pulses 170e-h.

Similar to that of the series of pulses 162 of waveform 160 (FIG. 7), in some examples, the series of electrical pulses 170 is a plurality of pulses including pairs of individual pulses that are coupled to one another. In particular, first pulse 170a is coupled with fifth pulse 170e, second pulse 170b is coupled with sixth pulse 170f, third pulse 170c is coupled with seventh pulse 170g, and fourth pulse 170d is coupled with eighth pulse 170h. As such, the temporal relationship between each pulse of a coupled pair of pulses is fixed. In the example shown in FIG. 9, the temporal relationship between each pulse of coupled pairs of pulses (e.g., pulses 170a and 170e, pulses 170b and 170f, and so forth) is such that the second pulse of the coupled pair (e.g., pulses 170e, 170f, 170g, and 170h) is delivered substantially immediately after the first pulse of the coupled pair (e.g., pulses 170a, 170b, 170c, and 170d, respectively) ends. In some examples, a fixed time delay may separate respective pulses of a coupled pair of pulses. For example, there may be a fixed delay of approximately 10 microseconds to approximately 100 microsecond between pulse 170a and pulse 170e. Such a fixed delay may be the same for each coupled pulse pair.

In some examples, series of pulses 170a-h may be substantially charged balanced. For example, first pulse 170a may have an equal and opposite charge from that of fifth pulse 170e. Notably, first pulse 170a and fifth pulse 170e may be charged balanced even though the amplitude of first pulse 170a is greater than that of fifth pulse 170e. To achieve substantial charge balance, the pulse width PW12 of fifth pulse 170e may be selected such that fifth pulse 170e extends front the trailing edge of first pulse 170a to the leading edge of second pulse 170b. Such timing may depend on the interval of time T(12) between start of first pulse 170a and second pulse 170b, in addition to the pulse width PW11 of first pulse 170a. The amplitude of fifth pulse 170e may be selected such that fifth pulse 170e has substantially the same charge as first pulse 170a when having a pulse width PW12. In such a case, fifth pulse 170e may have approximately the minimum amplitude allowed to maintain charge balance with first pulse 170a. For example, the area between the amplitude curve and the zero amplitude line for a first pulse 170a is approximately equal to the area between the amplitude curve and the zero amplitude line for the fifth pulse 170e.

In some examples, the difference in amplitude (current amplitude or voltage amplitude) of pulses 170a-d and pulses 170e-f may be such that the influence that the delivery of pulses 170a-d has on tissue at the target site is different than that of the influence that pulses 170e-f has on the same tissue at the target site. For example, pulses 170a-d may have a pulse amplitude that provides a pulse energy that is above a threshold required to depolarize one or/no/e cells at the target tissue site, while pulses 170e-g, which have a lower pulse amplitude from that of pulses 170a-d, may have a pulse amplitude that provides a pulse energy that is below that threshold required to depolarize the cells at the target tissue site. In such a situation, pulses 170e-h may provide for charge balanced stimulation without interfering with the physiological response of tissue to pulses 170a-d. By selecting the pulse width of pulse 170e-h to extend substantially from the end of the directly preceding pulse to the beginning of the following pulse, the amplitude of pulse 170e-h is minimized while allowing series of pulses 170a-h in be substantially charged balanced. In some examples, there may be a time delay between the second pulse of a coupled pair and the first pulse of the next coupled pair, e.g., between pulses 170e and 170b, although in such a case, the amplitude of pulse 170e is not minimized as described for some examples.

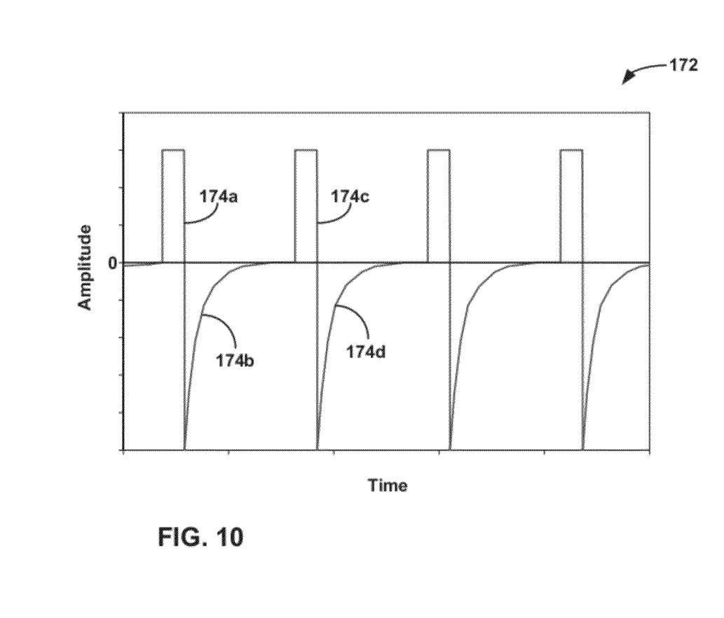

FIG. 10 is a plot illustrating another example waveform 172 representing an example series of electrical stimulation pulses for delivery to patient 16. Waveform 172 includes first pulse 174a, second pulse 174b, third pulse 174c, and fourth pulse 174d, which are delivered in direct succession with one another. As shown, first pulse 174a and third pulse 174c are rectangular pulses, and second pulse 174b and fourth pulse 174d are hyperbolic pulses. First pulse 174a and third pulse 174c have opposite polarity from that of second pulse 174b and fourth pulse 174d. IMD 12 delivers second pulse 174b substantially immediately after the end of first pulse 174a. Third pulse 174c is delivered to be substantially non-overlapping with second pulse 174b.

In some examples, first pulse 174a and second pulse 174 are coupled pulse pairs and third pulse 174c and fourth pulse 174d are coupled pulse pairs. As such, the temporal relationship between the delivery of first pulse 174a and second pulse 174b does not change as the frequency of the delivery of pulses 174a and 174c is changed. In the example shown, the fixed time interval between coupled pair pulses, e.g., between first pulse 174a and second pulse 174b, is approximately zero. In other examples, there may be a fixed time delay between approximately 10 microseconds and approximately 100 microseconds.

First pulse 174a has an approximately equal and opposite charge of that of second pulse 174b. As such first and second pulses 174a and 174b may be considered charge balanced with one another. In some examples, the shape of second pulse 174b reflects the passive recharge of capacitors in which stimulation generator 66 (FIG. 3) accumulates the charge to be delivered in third pulse 174c. The exact shape of second pulse 174b may vary with the amount of charge delivered in the first and third pulses 174a and 174c, in addition to the characteristics of the one or more capacitors of stimulation generator 66.

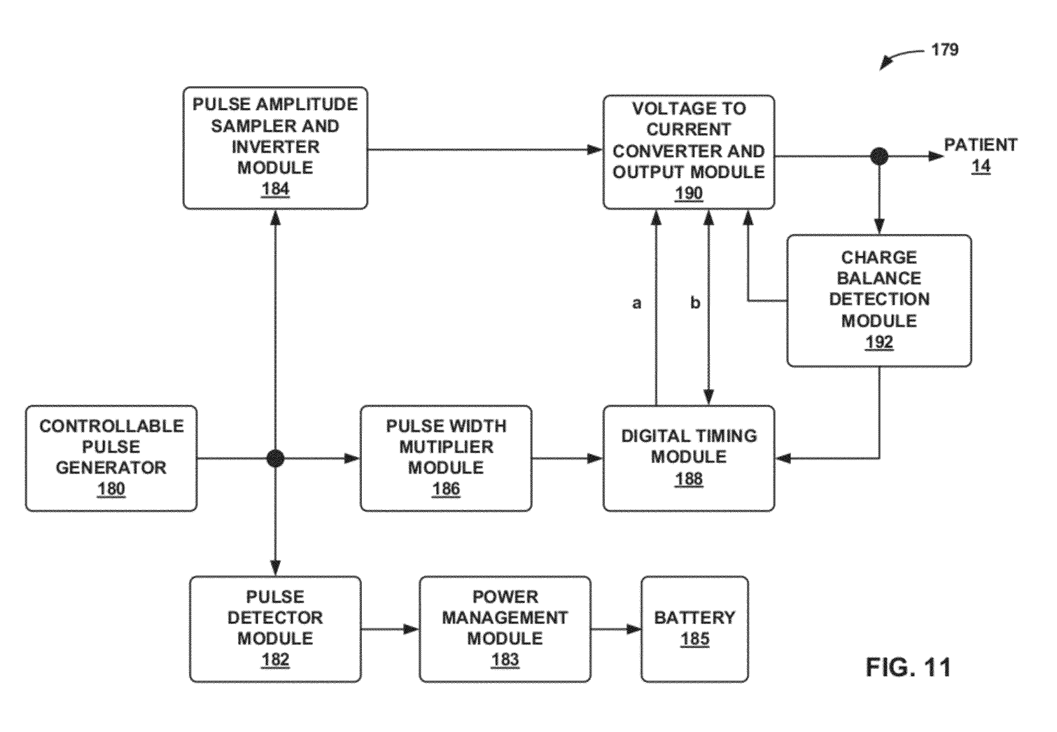

FIG. 11 is a block diagram of an example stimulation generator 179 that may be used to generate and deliver electrical stimulation therapy using one of more of the waveforms described in this disclosure. Stimulation generator 179 includes controllable pulse generator 180, pulse detector module 182, power management module 183, pulse amplitude sampler and inverter module 184, battery 185, pulse width multiplier module 186, digital timing module 188, voltage to current converter and output module 190, and charge balance module 192. The various components of FIG. 11 may be formed by any of a variety of discrete and/or integrated electrical circuitry, including logic circuitry such as one or more microprocessors, application specific integrated circuits (ASICs), field programmable gate arrays (FPGAs), or the like, or any combination of such circuitry.

Controllable pulse generator 180 generates an electrical signal waveform, which may comprise a series of electrical pulses modulated by a high frequency carrier signal and including controlled pulse amplitude, frequency, and width. In some examples, controllable pulse generator 180 may be a stimulation generator that is not capable of generating one or more pulses with the particular morphology desired for delivery to patient 14, e.g., based on the desired pulse width. As will be described below, in some examples, controllable pulse generator 180 may include a Medtronic Restore implantable pulse generator, manufactured by Medtronic, Inc. of Minneapolis, Minn., USA. As will be apparent from the following description, the parameters of the pulse train generated by controllable pulse generator 180 may be used to control the pulse width, pulse frequency, and pulse amplitude of the pulses generated by converter and output module 190.

The electrical signal waveform generated by controllable pulse generator 180 propagates to pulse detector module 182, pulse width multiplier module 186, and pulse amplitude sampler and inverter module 184. Power management module 183 may include power supply circuitry that generates one or more regulated supply voltages from power provided by battery 185. Power management module 183 may provide power to various components of stimulation generator 179 of FIG. 11, including voltage to current converter and output module 190. Pulse detector module 182 may include a pulse detector that detects pulses generated by controllable pulse generator 180. If pulse detector module 182 does not detect pulses from controllable pulse generator 180, then pulse detector module 182 may control power management module 183 to turn off power to one or more components of stimulation generator 179, such as voltage to current converter and output module 190, or enter a power conservation mode.

Pulse amplitude sampler and inverter module 184 detects the pulses generated by controllable pulse generator 180. Controllable pulse generator 180 generates the series of electrical pulses with a specific electrical pulse amplitude value, pulse width, and frequency, as well as carrier signal pulse amplitude, frequency and width, any or all of which may be selected by a user such as a physician or patient using an external programmer such as a external programmer or physician programmer. Pulse amplitude sampler and inverter module 184 may sample and measure the amplitude of the pulses generated by controllable pulse generator 180, and output a control signal and an inverted version of the control signal. The non-inverted and inverted control signals may have amplitude values proportional to the amplitude of the pulses generated by controllable pulse generator 180. In particular, based on the measured amplitude, pulse amplitude module 184 may generate non-inverted and inverted analog voltage signals which are received by voltage to current converter and output module 190 indicating the measured pulse amplitude.

Voltage to current converter and output module 190 receives the analog voltage signals from pulse amplitude sampler and inverter module 184, and selectively converts one of the voltage signals into current to generate a current pulse for delivery to patient 14. Digital timing module 188 controls voltage to current converter and output module 190 to output either a positive current pulse based on the non-inverted voltage signal or a negative current pulse based on the inverted voltage signal. For the example of an alternating monophasic waveform, e.g., as described with reference to FIG. 8, timing module 188 may control voltage to current converter and output module 190 to deliver positive and negative pulses on an alternating basis.

The amplitude of the current pulse is proportional to the amplitude of the inverted or non-inverted voltage signal, as applicable, that is provided by pulse amplitude sampler and inverter module 184. In turn, the amplitude of inverted or non-inverted voltage signal is proportional to the amplitude of the pulse obtained from controllable pulse generator 180. The pulse generated by controllable pulse generator 180 serves to control the current pulse generated by voltage to current converter and output module 190, which may act as a transconductance amplifier to convert voltage to current. As an illustration, if the amplitude of the pulse generated by pulse generator 180 is x volts, pulse amplitude sampler and inverter module 184 may generate inverted and non-inverted voltages representative of voltage level x. If the gain of voltage to current converter and output module 190 is y, then the output current pulse amplitude may be x*y amps.

As discussed above, module 190 generates electrical stimulation pulses with a pulse amplitude value that is defined based on the level of the analog voltage signal from pulse amplitude sampler and inverter module 184. Again, the level of the analog signal from pulse amplitude module 184 is proportional to the amplitude of the pulse from controllable pulse generator 180, which may be a controlled current or controlled voltage pulse. In this manner, the pulse amplitude (e.g., voltage pulse amplitude) generated by controllable pulse generator 180 may be used to specify the amplitude of a corresponding pulse (e.g., current pulse amplitude) to be generated by voltage to current converter and output module 190.

While pulse amplitude sampler and inverter module 184 controls the amplitude of the pulses to be delivered by voltage to current converter and output module 190, pulse width multiplier module 186 may determine the pulse width and frequency of the pulses, in conjunction with digital timing module 188. Pulse width multiplier module 186 detects the pulses generated by controllable pulse generator 180 having a controlled pulse width. Pulse width multiplier 186 may be configured to determine the pulse width of the received pulse and multiply that pulse width by a preset value n (e.g., n=5). Pulse width multiplier 186 then transmits a signal to digital timing module 188 indicating the calculated pulse width. As an example, assuming that pulse width multiplier module 186 is configured to multiply the pulse width of the signal from controllable pulse generator 180 by five, pulse width multiplier 186 may transmit a signal to timing module 188 indicating a puke width of 5 milliseconds upon the detection of a pulse generated by controllable pulse generator 180 having a pulse width of 1 millisecond.

Based on the pulse width value indicated by pulse width multiplier module 186, digital timing module 188 indicates to module 190 the timing for the delivery of pulses from module 190 to patient 14. For example, the timing may be expressed as a start and stop that defines a pulse width with a rising and falling edge. Timing module 190 also controls the polarity of the pulses delivered by module 190, e.g., by controlling the module to operate as either a current source or current sink. For a positive polarity pulse, for example, module 188 controls converter and output module 190 to operate as a current source using the non-inverted voltage from pulse amplitude sampler and inverter module 184 as an input signal. Conversely, for a negative polarity pulse, for example, module 188 controls converter and output module 190 to operate as a current sink using the in inverted voltage from pulse amplitude sampler and inverter module 184 as an input signal.

For the example of FIG. 8, digital timing module 188 may control output module 190 to deliver positive polarity pulses and negative polarity pulses on an alternating basis. To cause module 190 to deliver a positive pulse, module 188 may assert (e.g., enable or logic 1) and dessert (e.g., disable or logic 0) line a, where line a is asserted high for a period of time equal to the pulse width control signal generated by pulse width multiplier module 186 such that module 190 delivers the positive pulse for that period of time. To cause module 190 to deliver a negative pulse, module 188 may assert and dessert line b, where line a is asserted high for a period of time equal to the pulse width control signal generated by pulse width multiplier module 186 such that module 190 delivers the negative pulse for that period of time.

Module 190 may include parallel regulated current source and sink circuits that can be selectively activated to deliver positive and negative current pulses, respectively. The pulses may be delivered across a lead borne electrode and an electrode of the IMD housing, in a unipolar arrangement, or between two or more lead-borne electrodes in a multipolar arrangement. As an example, the source or sink of module 190 may be selectively activated by digital timing module 188 using signals applied via lines a and b. Again, the time for which a source or sink is activated may be a function of the pulse width indicated by pulse width multiplier module 186, which scales up the pulse width of the pulse generated by pulse generator 180 to provide a longer pulse width. Although the output of module 190 is described as current pulses for purposes of illustration, in some examples, stimulator generators applying principles of this disclosure may alternatively deliver voltage pulses.

Charge balance module 192 may be provided to monitor the output of module 190 to determine that the charge of the pulses delivered from module 190 to patient 14 is substantially balanced. For example, charge balance module 192 may detect a voltage across one of more output capacitors of module 190 to determine whether a charge imbalance remains following delivery of a stimulation pulse. Upon delivering a positive polarity pulse, for example, it may be desirable to restore the output voltage to a reference voltage, such as zero volts. For some waveforms, charge balance module 192 may transmit a signal to digital timing module 188 to indicate instances of imbalanced charge, and cause digital timing module 188 to adjust the pulse width of a negative polarity pulse such that it is truncated or shortened to provide a substantial charge balance with respect to a positive polarity pulse. In this case, digital timing module 188 may control pulse width based on the input of module 186 and module 192. For other waveforms, charge balance module 192 may transmit a signal to module 190 to indicate instances of imbalanced charge and cause module 190 to reduce or increase an amplitude of the a negative polarity pulse such that it is increased or decreased to provide a substantial charge balance with respect to a positive polarity pulse. For some waveforms, charge balance module 192 may control both digital timing module 188 and module 190 to adjust pulse width and amplitude of a negative polarity pulse to provide a substantial charge balance with respect to a positive polarity pulse.

Stimulation generator 179 is only one example of a stimulation generator that may be used to generate and deliver stimulation pulses to a patient in a manner consistent with one or more waveforms described in this disclosure. Other example stimulation pulse generators capable of generating and delivering pulses with the desired morphology are contemplated. In other examples, a current regulator may be controlled to operate as a regulated current source or sink and to deliver current pulses with desired polarity, frequency and pulse width to provide any of the waveforms described in this disclosure. Alternatively, a voltage-based stimulation generator may be provided in other examples to delivery controlled voltage pulses instead of controlled current pulses. Accordingly, the example arrangement of FIG. 11 is provided for purposes of illustration as one example of the convenient use of a controllable pulse generator with one set of capabilities (such as pulse width) to drive and control another pulse generator to provide another set of capabilities (e.g., larger pulses widths). In the example of FIG. 11, the first pulse generator 180 has parameters that are controlled to, in turn, control the components of stimulation generator 179 as described. However, a single pulse generator (or multiple pulse generators provided for multiple electrodes) may be used.

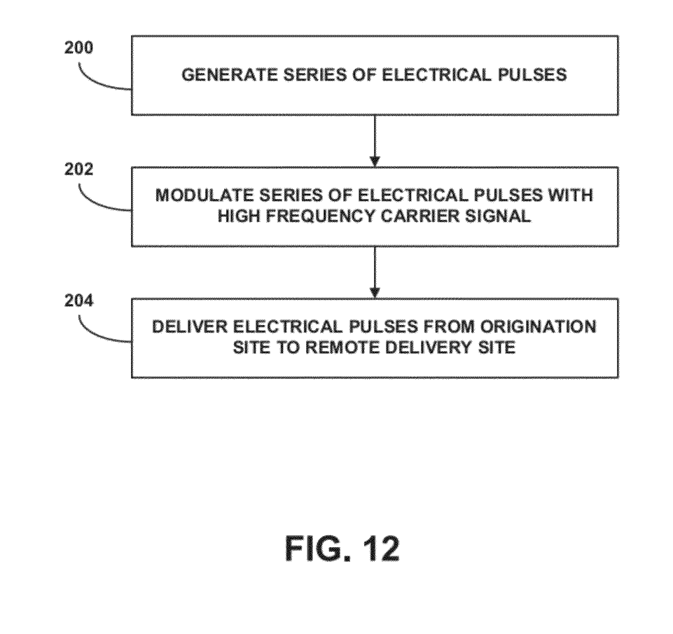

FIG. 12 is a flowchart illustrating an example method according to this disclosure. The example method of FIG. 12 includes generating a series of electrical pulses comprising a first frequency and alternating pulse polarities (200), modulating the series of electrical pulses with a carrier signal comprising a second frequency such that each pulse in the series of electrical pulses comprises a series of carrier pulses comprising a single polarity and the second frequency (202), and delivering the series of electrical pulses modulated by the high frequency carrier signal from an origination site to a remote delivery site within a patient (204).

The functions of the method of FIG. 12 for generating and delivering a series of charge balanced electrical pulses modulated by a high frequency carrier signal to a patient are described as carried out by various components of therapy system 10 of FIG. 1. However, in other examples, one or more of these functions may be carried out by other devices including, e.g., devices associated with the system described with reference to FIG. 2. For example, instead of delivering a series of charge balanced electrical pulses modulated by a high frequency carrier signal to a patient subcutaneously according to the example of FIG. 1, in one example, the series of charge balanced electrical pulses modulated by a high frequency carrier signal may be delivered to the patient transcutaneously according to the example of FIG. 2.

The method of FIG. 12 includes generating a series of electrical pulses comprising a first frequency and alternating pulse polarities (200). In one example, processor 60 controls stimulation generator 66 to generate a series of electrical pulses including a first frequency and alternating pulse polarities (200), e.g. according to one or more programs stored in memory 62 of IMD 16. In one example, processor 60 controls stimulation generator 66 to generate stimulation waveforms characterized by parameters and values thereof described in examples according to this disclosure. For example, processor 60 may control stimulation generator 66 to generate a waveform designed to penetrate tissue within patient 14 to transmit electrical stimulation from an origination site to remote delivery site within the patient. In one example, processor 60 employs a program stored in memory 62 to control stimulation generator 66 to generate a series of alternating polarity electrical pulses including a first frequency in a range from approximately 4 hertz (Hz) to approximately 100 Hz. In another example, processor 60 controls stimulation generator 66 to generate a series of alternating polarity electrical pulses including a first frequency in a range from approximately 5 Hz to approximately 25 Hz. In another example, processor 60 controls stimulation generator 66 to generate a series of alternating polarity electrical pulses including a first frequency in a range from approximately 5 Hz to approximately 14 Hz. In another example, processor 60 controls stimulation generator 66 to generate a series of alternating polarity electrical pulses including a first frequency approximately equal to 80 Hz.

The series of electrical pulses generated by stimulation generator 66 may also be characterized by a number of parameters other than the first frequency, including, e.g. amplitude and pulse width. In one example, processor 60 may employ a program stored on memory 62 that specifies one or more values for such parameters, including specific parameters for the amplitude and pulse width of the series of electrical pulses generated by stimulation generator 66. In one example, processor 60 employs a program stored on memory 62 that specifies values for a substantially constant voltage amplitude of the series of electrical pulses generated by stimulation generator 66 in a range from greater than zero to less than or equal to approximately 25 volts. In another example, processor 60 employs a program stored on memory 62 that specifies values for a substantially constant current amplitude of the series of electrical pulses generated by stimulation generator 66 in a range from greater than zero to less than or equal to approximately 25 milliamps. In one example, processor 60 employs a program stored on memory 62 that specifies values for a the pulse width of one or more pulses in the series of electrical pulses generated by stimulation generator 66 in a range from approximately 100 μs to approximately 5 ms.

Whatever the frequency, amplitude, pulse width, and any other parameters of the series of electrical pulses generated by stimulation generator 66, processor may control the generator to generate substantially charge balanced successive electrical pulses such that the net charge of each successive pair of pulses is approximately equal to zero. In an example in which processor 60 controls stimulation generator 66 to generate a series of biphasic electrical pulses, each coupled pair of pulses in a biphasic pulse may include approximately equal charges of opposite polarity. As such, the net charge of the coupled pair of pulses in the biphasic pulse is approximately equal to zero. In an example in which processor 60 controls stimulation generator 66 to generate a series of monophasic electrical pulses, each pair of successive monophasic pulses may include approximately equal charges of opposite polarity. As such, the net charge of the successive pair of monophasic pulses is approximately equal to zero.

The method of FIG. 12 also includes modulating the series of electrical pulses with a carrier signal comprising a second frequency such that each pulse in the series of electrical pulses comprises a series of carrier pulses comprising a single polarity and the second frequency (202), in one example, processor 60 may control stimulation generator 66 to modulate the series of electrical stimulation pulses with a high frequency carrier signal. In another example, processor 60 may control a component separate from stimulation generator 66 that functions to modulate the series of electrical stimulation pulses with a high frequency carrier signal. Other component and functional configurations for modulating the series of electrical stimulation pulses with a high frequency carrier signal are also contemplated. For example, a pulse modulation module separate from stimulation generator may function autonomously; e.g. not under the control of another component like processor 60, to modulate the series of electrical stimulation pulses with a high frequency carrier signal.

In one example, a program employed by processor 60 may include one or more values for the second frequency of the carrier signal to enable the series of electrical pulses modulated by the carrier signal and delivered by stimulation generator 66 to penetrate tissue of patient 14 between an origination site and a remote delivery site. In one example, processor 60 employs a program stored in memory 62 to control stimulation generator 66 to modulate the series of electrical pulses with a carrier signal including a second frequency in a range from approximately 1 kilohertz (kHz) to approximately 500 kHz. In one example, processor 60 employs a program stored in memory 62 to control stimulation generator 66 to modulate the series of electrical pulses with a carrier signal including a second frequency in a range from approximately 4 kilohertz (kHz) to approximately 400 kHz. In one example, processor 60 employs a program stored in memory 62 to control stimulation generator 66 to modulate the series of electrical pulses with a carrier signal including a second frequency approximately equal to 200 kHz.

The program or programs by which processor 60 functions to control stimulation generator 66 to modulate the series of electrical pulses with a high frequency carrier signal may include pulse width values for the carrier pulses of the high frequency carrier signal. The pulse width of the carrier pulses may be, in some examples, set as a function of the frequency of the carrier signal. For example, a carrier signal with a frequency of 100 kHz may include pulses with pulse widths in a range from greater than zero but less than or equal to approximately 1/100,000 seconds or 10 microseconds (μs). In another example, a carrier signal with a frequency of 200 kHz may include pulses with pulse widths in a range from greater than zero but less than or equal to approximately 1/200,000 seconds or 200 milliseconds (μs).

In addition to modulating the series of electrical pulses with a carrier signal comprising a second frequency such that each pulse in the series of electrical pulses comprises a series of carrier pulses comprising a single polarity and the second frequency (202), the method of FIG. 12 also includes delivering the series of electrical pulses modulated by the high frequency carrier signal from an origination site to a remote delivery site within a patient (204). In one example, processor 60 controls stimulation generator 66 to deliver the series of electrical pulses modulated by the high frequency carrier signal subcutaneously from an origination site inside of the body of patient 14, e.g. site 30 in the example of FIG. 1, to a remote delivery site within the patient, e.g. site 32 in FIG. 1. In one example, processor 60 controls stimulation generator 66 to deliver the series of electrical pulses modulated by the high frequency carrier signal transcutaneously from an origination site outside of the body of patient 14, e.g. site 58 in the example of FIG. 2, to a remote delivery site within the patient, e.g. site 60 in FIG. 2. Remote delivery sites within patient 14 may include, e.g., spinal, sacral or pudendal nerve, e.g. to reduce a frequency of contractions of bladder 12, as well as a hypogastric nerve, a pudendal nerve, a dorsal penile/clitoral nerve, the urinary sphincter, or any combination thereof to a promote closure of a urinary sphincter of patient 14.

In one example according to this disclosure, IMD 16 may be coupled to a percutaneous lead including electrodes arranged at the distal end of the lead within the body of patient 14. In such an example, processor 60 may be configured to control stimulation generator 66 to deliver remote electrical stimulation to patient 14 via series of electrical pulses modulated by the high frequency carrier signal delivered by the implanted electrodes at the distal end of the percutaneous lead from the origination site within the body of the patient to a remote delivery site within the body, e.g. at or near one or more pelvic floor nerves.

While examples of the disclosure are generally described with regard to pelvic floor conditions of a patient and, in particular, the delivery of stimulation therapy to pelvic floor nerves to treat a urinary tract condition. Some examples of this disclosure may apply to other types of stimulation therapy in a manner that effectively treats a patient condition other than that of a urinary tract or other pelvic floor condition. In some examples, such electrical stimulation may be generated and delivered to provide for electrical stimulation of one or more patient nerve structures or sites. For example, a medical device may generate and deliver spinal cord stimulation (SCS), peripheral nerve stimulation, and/or peripheral nerve field stimulation (PNFS) in accordance with one or more of the examples described herein. In one example, stimulation may be delivered to the spinal cord to treat neuropathic pain. In another example, stimulation may be delivered to the vagus nerve tier treatment of eating disorders, anxiety, schizophrenia, depression, epilepsy, or hormonal disorders. In another example, stimulation may be delivered to the hypoglossal nerve for treatment of sleep apnea. In another example, diaphragm stimulation may be provided, for example, to manage respiration of a patient. For example, such stimulation may be delivered to the phrenic nerve of a patient to induce or otherwise manage respiration of a patient.

As another example, a medical device may generate and deliver electrical stimulation to other non-urinary tract body organs including, e.g., heart, liver, pancreas, kidney, and/or blood vessels, in accordance with one or more examples described herein. In one example, stimulation may be delivered to the heart for cardiac pacing for treatment of brachycardia.

In each case described above, the electrical stimulation therapy may be configured to effectively treat one or more patient conditions associated with the particular type of stimulation therapy.

The techniques described in this disclosure for generating and delivering series of charge balanced electrical pulses modulated by a high frequency carrier signal may be implemented in hardware, software, firmware or any combination thereof. For example, various aspects of the techniques may be implemented within one or more microprocessors, digital signal processors (DSPs), application specific integrated circuits (ASICs), field programmable logic arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic media, optical media, or the like. The instructions are executed to support one or more aspects of the functionality described in this disclosure.

Various aspects of the disclosure have been described. These and other aspects are within the scope of the following claims.

1. A method comprising:
generating a series of electrical pulses comprising a first frequency and alternating pulse polarities;
modulating the series of electrical pulses with a carrier signal comprising a second frequency such that each pulse in the series of electrical pulses comprises a series of carrier pulses comprising a single polarity and the second frequency; and
delivering the series of electrical pulses modulated by the carrier signal from an origination site to a remote delivery site within a patient,
wherein the first frequency is configured to produce a therapeutic effect and the second frequency is configured to enable the series of electrical pulses to penetrate tissue between the origination site and the remote delivery site, and
wherein the series of electrical pulses is substantially charge balanced.

2. The method of claim 1, wherein the second frequency is at least an order of magnitude larger than the first frequency.

3. The method of claim 2, wherein the first frequency is in a range from approximately 4 hertz (Hz) to approximately 100 Hz.

4. The method of claim 2, wherein the first frequency is approximately equal to 80 Hz.

5. The method of claim 2, wherein the first frequency is in a range from approximately 5 Hz to approximately 25 Hz.

6. The method of claim 5, wherein the first frequency is in a range from approximately 5 Hz to approximately 14 Hz.

7. The method of claim 1, wherein the second frequency is in a range from approximately 1 kilohertz (kHz) to approximately 500 kHz.

8. The method of claim 7, wherein the second frequency is in a range from approximately 4 kHz to approximately 400 kHz.

9. The method of claim 8, wherein the second frequency is approximately equal to 200 kHz.

10. The method of claim 1, wherein each pulse in the series of electrical pulses comprises a pulse width in a range from approximately 100 microseconds (is) to approximately 5 milliseconds.

11. The method of claim 1, wherein each pulse in the series of electrical pulses comprises a pulse width in a range from approximately 200 microseconds (μs) to approximately 1 milliseconds.

12. The method of claim 1, wherein each carrier pulse comprises a pulse width approximately equal to one divided by the second frequency.

13. The method of claim 1, wherein delivering the series of electrical pulses comprises delivering each pulse in the series of electrical pulses with a controlled voltage and an amplitude greater than zero but less than or equal to approximately 25 volts.

14. The method of claim 1, wherein delivering the series of electrical pulses comprises delivering each pulse in the series of electrical pulses with a controlled current and an amplitude greater than zero but less than or equal to approximately 25 milliamps.

15. The method of claim 1, wherein each pair of successive pulses in the series of electrical pulses comprises a first pulse and a second pulse, wherein the second pulse comprises a polarity opposite a polarity of the first pulse and at least a pulse width, an amplitude, and a carrier signal frequency approximately equal to a pulse width, amplitude, and carrier signal frequency of the first pulse.

16. The method of claim 1, wherein delivering the series of electrical pulses from the origination site to the remote delivery site within the patient comprises delivering the series of electrical pulses transcutaneously from outside of a body of the patient to the remote delivery site within the patient.

17. The method of claim 1, wherein delivering the series of electrical pulses from the origination site to the remote delivery site within the patient comprises delivering the series of electrical pulses subcutaneously from an origination site within the patient to the remote delivery site within the patient.

18. The method of claim 18, wherein each pair of successive pulses in the series of electrical pulses is substantially completely charged balanced.

19. A medical system comprising:
a stimulation generator configured to generate and deliver electrical pulses from an origination site to a remote delivery site within a patient; and
a processor configured to control the stimulation generator to generate and deliver a series of electrical pulses comprising a first frequency and alternating pulse polarities and modulate the series of electrical pulses with a carrier signal comprising a second frequency such that each pulse in the series of electrical pulses comprises a series of carrier pulses comprising a single polarity and the second frequency,
wherein the first frequency is configured to produce a therapeutic effect and the second frequency is configured to enable the series of electrical pulses to penetrate tissue between the origination site and the remote delivery site, and
wherein the series of electrical pulses is substantially charged balanced.

20. The system of claim 19, further comprising a medical lead connected to the stimulation generator and comprising one or more electrodes configured to deliver the series of electrical pulses to the remote delivery site, wherein the electrodes are configured to be located at the origination site.

21. The system of claim 19, wherein the one or more electrodes comprise one or more epidermal electrodes configured to deliver the series of electrical pulses transcutaneously from outside of a body of the patient to the remote delivery site within the patient.

22. The system of claim 19, wherein the medical lead comprises a percutaneous lead configured to arrange the one or more electrodes at an origination sight within a body of the patient, and wherein the one or more electrodes are configured to deliver the series of electrical pulses subcutaneously from the origination site within the patient to the remote delivery site within the patient.

23. The system of claim 19, wherein the first frequency is in a range from approximately 4 hertz (Hz) to approximately 100 Hz.

24. The system of claim 19, wherein the second frequency is in a range from approximately 1 kilohertz (kHz) to approximately 500 kHz.

25. The system of claim 19, wherein each pulse in the series of electrical pulses comprises a pulse width in a range from approximately 100 microseconds (μs) to approximately 5 milliseconds.

26. The system of claim 19, wherein each carrier pulse comprises a pulse width approximately equal to one divided by the second frequency.

27. The system of claim 19, wherein the processor is configured to control the stimulation generator to generate and deliver each pulse in the series of electrical pulses with a controlled voltage and an amplitude greater than zero but less than or equal to approximately 25 volts.

28. The system of claim 19, wherein the processor is configured to control the stimulation generator to generate and deliver each pulse in the series of electrical pulses with a controlled current and an amplitude greater than zero but less than or equal to approximately 25 milliamps.

29. The system of claim 19, wherein each pair of successive pulses in the series of electrical pulses is substantially completely charged balanced.

30. A non-transitory computer-readable storage medium comprising instructions to cause a programmable processor to:
control a stimulation generator to generate a series of electrical pulses comprising a first frequency and alternating pulse polarities, modulate the series of electrical pulses with a carrier signal comprising a second frequency such that each pulse in the series of electrical pulses comprises a series of carrier pulses comprising a single polarity and the second frequency, and deliver the series of electrical pulses modulated by the carrier signal from an origination site to a remote delivery site within a patient,
wherein the first frequency is configured to produce a therapeutic effect and the second frequency is configured to enable the series of electrical pulses to penetrate tissue between the origination site and the remote delivery site, and
wherein the series of electrical pulses is substantially charged balanced.

31. A medical system comprising:
means for generating a series of electrical pulses comprising a first frequency and alternating pulse polarities;
means for modulating the series of electrical pulses with a carrier signal comprising a second frequency such that each pulse in the series of electrical pulses comprises a series of carrier pulses comprising a single polarity and the second frequency; and
means for delivering the series of electrical pulses modulated by the carrier signal from an origination site to a remote delivery site within a patient,
wherein the first frequency is configured to produce a therapeutic effect and the second frequency is configured to enable the series of electrical pulses to penetrate tissue between the origination site and the remote delivery site, and
wherein the series of electrical pulses is substantially charge balanced.
